(19) United States
(12) Patent Application Publication
(10) Pub. No.: US 2026/0193184 A1
(43) Pub. Date: Jul. 9, 2026

(54) STEREOGENIC 3,3-DISUBSTITUTED 3-AMINO OXINDOLE COMPOUNDS AND A PROCESS FOR PREPARATION THEREOF

(71) Applicant: COUNCIL OF SCIENTIFIC & INDUSTRIAL RESEARCH, New Delhi (IN)

(72) Inventors: Benudhar PUNJI, Pune (IN); Shidheshwar Baliram ANKADE, Pune (IN); Chandini PRADHAN, Pune (IN)

(21) Appl. No.: 19/132,208

(22) PCT Filed: Nov. 25, 2023
(86) PCT No.: PCT/IN2023/051098

§ 371 Date: May 22, 2025

(30) Foreign Application Priority Data
Nov. 25, 2022 (IN) 202211068206

(51) Int. Cl. C07D 209/40; C07B 59/00; C07D 405/14; C07J 43/00

(52) U.S. Cl. CPC C07D 209/40; C07B 59/002; C07D 405/14; C07J 43/003; C07B 2200/05

(57) ABSTRACT

The present invention provides stereogenic 3,3-disubstituted 3-aminooxindole compounds of formula (I). In addition, the present invention provides a process for preparing a stereogenic 3,3-disubstituted 3-aminooxindole compound of formula (I) using isatins and N-methoxy benzamides in presence of highly abundant iron catalyst. The stereogenic 3,3-disubstituted 3-aminooxindole compound of formula (I) is useful in the preparation of various bio-relevant C3-aminated oxindoles.

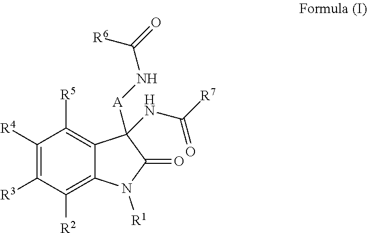

### FIELD OF THE INVENTION

The present invention relates to a stereogenic 3,3-disubstituted 3-aminooxindole compounds of formula (I). Particularly, the present invention relates to a process for generating quaternary stereogenic carbon center. More particularly, the present invention provides an efficient process for the synthesis of stereogenic 3,3-disubstituted 3-aminooxindoles compound of formula I by iron-catalyzed reaction of isatins with benzamides. The present invention further provides a process for preparation of bio-relevant C3-aminated oxindoles using the stereogenic 3,3-disubstituted 3-aminooxindole compound of formula (I).

### BACKGROUND OF THE INVENTION

An oxindole scaffold containing quaternary stereogenic center at the C3 position, such as 3,3-disubstituted 3-aminooxindole, is present in various biologically important compounds and natural products. Such 3,3-disubstituted-3-aminooxindoles are obtained by multi-steps process involving addition of sensitive nucleophiles to isatin ketimines.

Reference may be made to Journal “Adv. Synth. Catal. 2010, 352, 1381-1407”, reveals the 3,3′-disubstituted oxindole structural motif is a prominent feature in many alkaloid natural products, which include all kinds of tetra substituted carbon stereocenters, spirocyclic or not, all-carbon or heteroatom-containing. The catalytic asymmetric synthesis of the tetrasubstituted carbon stereocenter at the C-3 position of the oxindole framework integrates new synthetic methods and chiral catalysts, reflects the latest achievements in asymmetric catalysis, and facilitates the synthesis of sufficient quantities of related compounds as potential medicinal agents and biological probes. Some of the biologically active 3-substituted 3-amino-2-oxindoles are as follows:

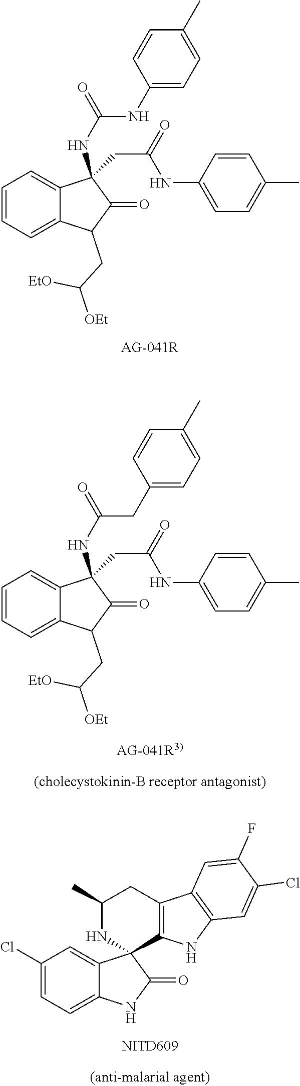

Reference may be made to Journal “Xu Cheng et al., J. Am. Chem. Soc. 2008, 130, 47, 15786-15787”, which reports the synthesis of a 3,3-diamino-substituted spirocyclic oxoindole compound with 84% ee, by the reaction of isatin with 2-aminobenzamide in the presence of a chiral phosphoric acid derivative ((S)-TRIP) as a catalyst. This protocol involves in generating a C3 quaternary carbon center with two —NHR units forming a cyclic structures (spirocycle), which are significantly different than the present invention. Moreover, the protocol of Xu Cheng et al uses an aldehyde derivative for the reaction, which is different in the present application (amide).

Reference may be made to Patent Application “CN104693092A” published on 16 Feb. 2015 reports a diastereo- and enantioselective synthetic method of chiral 3,3-disubstituted oxoindole derivatives using diazoisatin, indole, arylamine and aldehyde ester as raw materials, a metal catalyst which can be an iron-based catalyst, chiral phosphoric acid as a co-catalyst, an organic solvent, and molecular sieve as an additive, in a one-step reaction performed at 25° C. The protocol and compound prepared therein of this CN patent recites a C3 quaternary carbon center with —CHRNHR and -heterocycle linked to quaternary center. However, the present application is different in terms of a quaternary carbon center with —NHR and —CH₂—NHR units. These acyclic amino and aminomethyl units generate different chemical features and can serve as a unique precursor for further development. Moreover, the protocol of CN patent uses a protected diazoisatin that involved additional steps for the synthesis, thus increasing overall cost and purification steps, hence, not preferable.

### OBJECTIVES OF THE INVENTION

Main objective of the present invention is to provide a stereogenic 3,3-disubstituted 3-aminooxindole compound of formula (I).

Another objective of the present invention is to provide an efficient method for the synthesis of quaternary stereogenic center at C3 position of oxindoles.

Another objective of the present invention is to provide a process of preparation of 3,3-disubstituted 3-aminooxindoles compound of formula (I) using isatins and benzamides in the presence of highly abundant iron catalyst.

Yet another objective of the present invention is to provide a stereogenic 3,3-disubstituted 3-aminooxindoles compound of formula (I) for the preparation of bio-relevant C3-aminated oxindoles.

### SUMMARY OF THE INVENTION

Accordingly, the present invention provides a stereogenic 3,3-disubstituted 3-aminooxindole compounds of formula (I). The present invention further relates to a process for generating quaternary stereogenic carbon center by providing an efficient process for the synthesis of stereogenic 3,3-disubstituted 3-aminooxindoles by iron-catalyzed reaction of isatins with benzamides.

Accordingly, present invention provides a stereogenic 3,3-disubstituted 3-aminooxindole compound of formula (I)

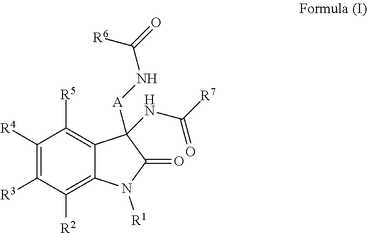

or isomer, solvate, and tautomer form thereof,
wherein
A is —CHR—, or —CD₂;
R is hydrogen, or unsubstituted or substituted C₁₋₆ alkyl;
R¹ is unsubstituted or substituted C₁₋₈ alkyl, unsubstituted or substituted aryl, unsubstituted or substituted alkylaryl, unsubstituted or substituted cycloalkyl, unsubstituted or substituted alkylalkoxy, unsubstituted or substituted alkoxyaryl, unsubstituted or substituted heteroaryl, unsubstituted or substituted alkylheteroaryl, unsubstituted or substituted alkoxyheteroaryl, unsubstituted or substituted ester, unsubstituted or substituted alkyloxy-cycloalkenealkyl, unsubstituted or substituted alkyloxy-aryl, unsubstituted or substituted alkyloxy-heteroaryl, unsubstituted or substituted alkyloxy-arylalkene, or unsubstituted or substituted alkene,
R² is hydrogen, halogen, unsubstituted or substituted C₁₋₆ alkyl, unsubstituted or substituted aryl, or unsubstituted or substituted heteroaryl,
R³ is hydrogen, halogen, unsubstituted or substituted C₁₋₆alkyl, unsubstituted or substituted aryl, or unsubstituted or substituted heteroaryl,
R⁴ is hydrogen, halogen, unsubstituted or substituted C₁₋₆alkyl, unsubstituted or substituted alkoxy, unsubstituted or substituted aryl, or unsubstituted or substituted heteroaryl,
R⁵ is hydrogen, halogen, unsubstituted or substituted C₁₋₆alkyl, unsubstituted or substituted aryl, or unsubstituted or substituted heteroaryl,
R⁶ is unsubstituted or substituted aryl, or unsubstituted or substituted heteroaryl, and
R⁷ is unsubstituted or substituted aryl, or unsubstituted or substituted heteroaryl.

In an embodiment of the present invention, the compound is selected from the group consisting of:

| Compound | |
|---|---|
| No. | Chemical Structure |
| 1 | 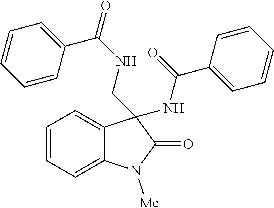 [SMILES: CN1C(=O)C(CNC(=O)c2ccccc2)(NC(=O)c2ccccc2)c2ccccc21] |
| 2 | 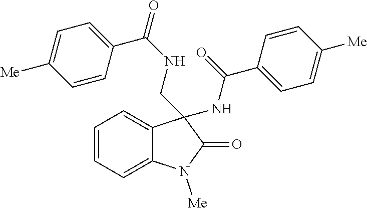 [SMILES: Cc1ccc(C(=O)NCC2(NC(=O)c3ccc(C)cc3)C(=O)N(C)c3ccccc32)cc1] |
| 3 | 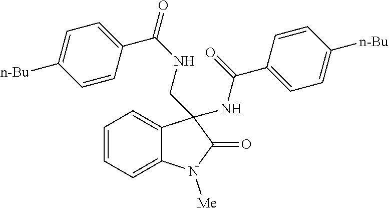 |
| 4 | 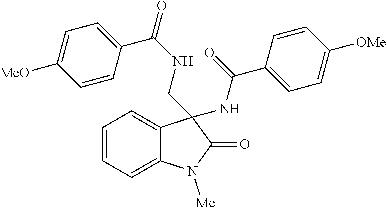 [SMILES: COc1ccc(C(=O)NCC2(NC(=O)c3ccc(OC)cc3)C(=O)N(C)c3ccccc32)cc1] |
| 5 | 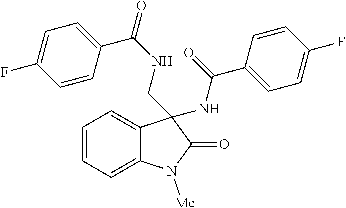 [SMILES: CN1C(=O)C(CNC(=O)c2ccc(F)cc2)(NC(=O)c2ccc(F)cc2)c2ccccc21] |
| 6 | 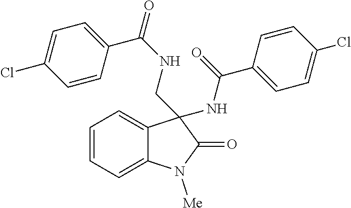 [SMILES: CN1C(=O)C(CNC(=O)c2ccc(Cl)cc2)(NC(=O)c2ccc(Cl)cc2)c2ccccc21] |
| 7 | 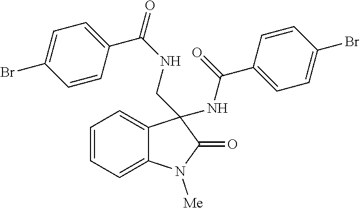 [SMILES: CN1C(=O)C(CNC(=O)c2ccc(Br)cc2)(NC(=O)c2ccc(Br)cc2)c2ccccc21] |
| 8 | 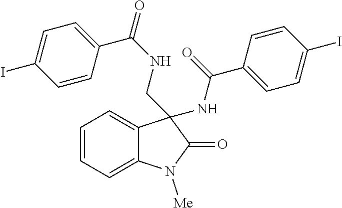 [SMILES: CN1C(=O)C(CNC(=O)c2ccc(I)cc2)(NC(=O)c2ccc(I)cc2)c2ccccc21] |
| 9 | 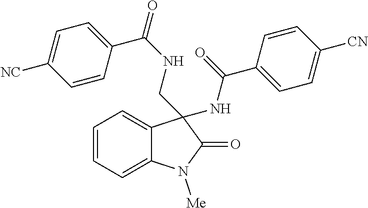 [SMILES: CN1C(=O)C(CNC(=O)c2ccc(C#N)cc2)(NC(=O)c2ccc(C#N)cc2)c2ccccc21] |
| 10 | 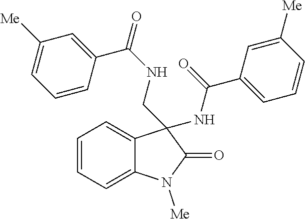 [SMILES: Cc1cccc(C(=O)NCC2(NC(=O)c3cccc(C)c3)C(=O)N(C)c3ccccc32)c1] |
| 11 | 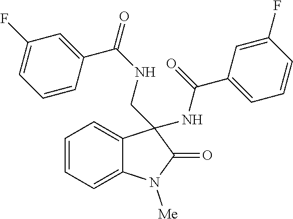 [SMILES: CN1C(=O)C(CNC(=O)c2cccc(F)c2)(NC(=O)c2cccc(F)c2)c2ccccc21] |
| 12 | 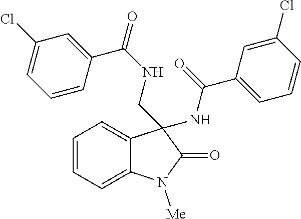 [SMILES: CN1C(=O)C(CNC(=O)c2cccc(Cl)c2)(NC(=O)c2cccc(Cl)c2)c2ccccc21] |
| 13 | 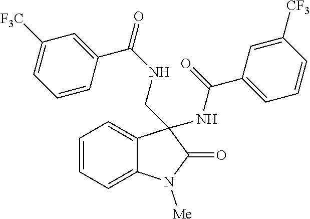 [SMILES: CN1C(=O)C(CNC(=O)c2cccc(C(F)(F)F)c2)(NC(=O)c2cccc(C(F)(F)F)c2)c2ccccc21] |
| 14 | 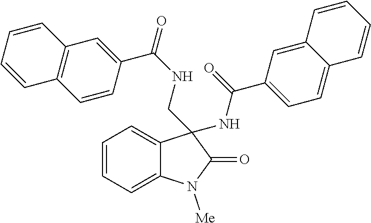 [SMILES: CN1C(=O)C(CNC(=O)c2ccc3ccccc3c2)(NC(=O)c2ccc3ccccc3c2)c2ccccc21] |
| 15 | 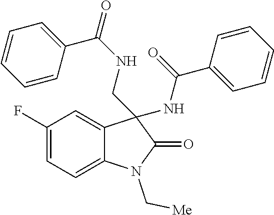 [SMILES: CCN1C(=O)C(CNC(=O)c2ccccc2)(NC(=O)c2ccccc2)c2cc(F)ccc21] |
| 16 | 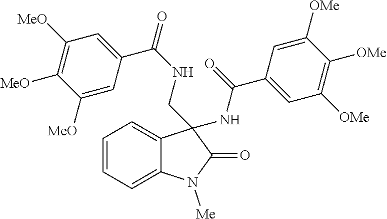 [SMILES: COc1cc(C(=O)NCC2(NC(=O)c3cc(OC)c(OC)c(OC)c3)C(=O)N(C)c3ccccc32)cc(OC)c1OC] |
| 17 | 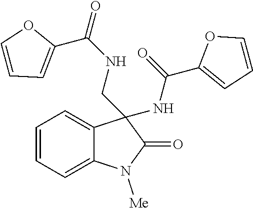 [SMILES: CN1C(=O)C(CNC(=O)c2ccco2)(NC(=O)c2ccco2)c2ccccc21] |
| 18 | 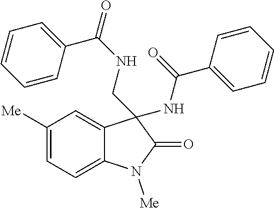 [SMILES: Cc1ccc2c(c1)C(CNC(=O)c1ccccc1)(NC(=O)c1ccccc1)C(=O)N2C] |
| 19 | 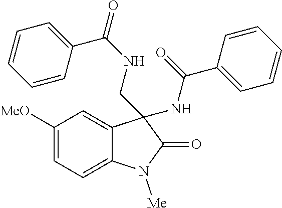 [SMILES: COc1ccc2c(c1)C(CNC(=O)c1ccccc1)(NC(=O)c1ccccc1)C(=O)N2C] |
| 20 | 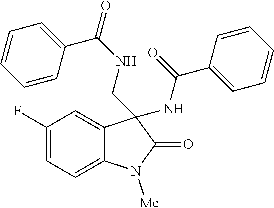 [SMILES: CN1C(=O)C(CNC(=O)c2ccccc2)(NC(=O)c2ccccc2)c2cc(F)ccc21] |
| 21 | 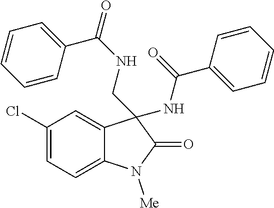 [SMILES: CN1C(=O)C(CNC(=O)c2ccccc2)(NC(=O)c2ccccc2)c2cc(Cl)ccc21] |
| 22 | 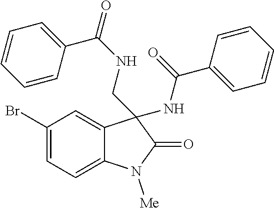 [SMILES: CN1C(=O)C(CNC(=O)c2ccccc2)(NC(=O)c2ccccc2)c2cc(Br)ccc21] |
| 23 | 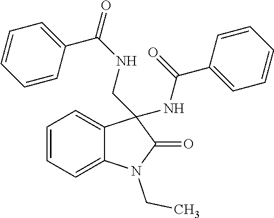 [SMILES: CCN1C(=O)C(CNC(=O)c2ccccc2)(NC(=O)c2ccccc2)c2ccccc21] |
| 24 | 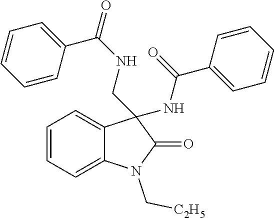 [SMILES: CCCN1C(=O)C(CNC(=O)c2ccccc2)(NC(=O)c2ccccc2)c2ccccc21] |
| 25 | 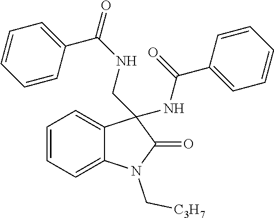 |
| 26 | 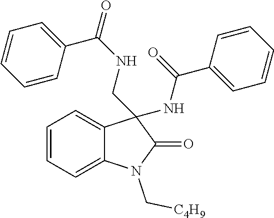 |
| 27 | 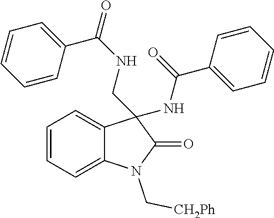 |
| 28 | 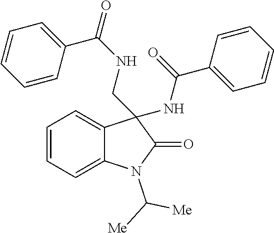 [SMILES: CC(C)N1C(=O)C(CNC(=O)c2ccccc2)(NC(=O)c2ccccc2)c2ccccc21] |
| 29 | 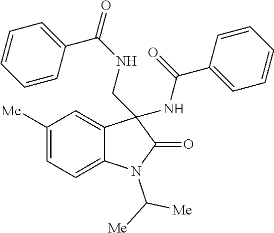 [SMILES: Cc1ccc2c(c1)C(CNC(=O)c1ccccc1)(NC(=O)c1ccccc1)C(=O)N2C(C)C] |
| 30 | 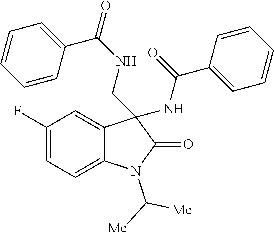 [SMILES: CC(C)N1C(=O)C(CNC(=O)c2ccccc2)(NC(=O)c2ccccc2)c2cc(F)ccc21] |
| 31 | 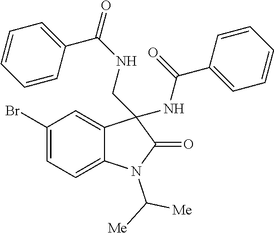 [SMILES: CC(C)N1C(=O)C(CNC(=O)c2ccccc2)(NC(=O)c2ccccc2)c2cc(Br)ccc21] |
| 32 | 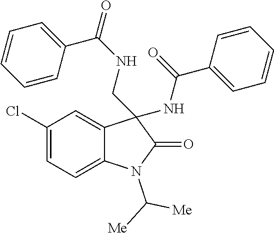 [SMILES: CC(C)N1C(=O)C(CNC(=O)c2ccccc2)(NC(=O)c2ccccc2)c2cc(Cl)ccc21] |
| 33 | 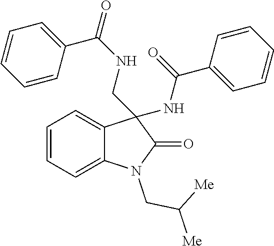 [SMILES: CC(C)CN1C(=O)C(CNC(=O)c2ccccc2)(NC(=O)c2ccccc2)c2ccccc21] |
| 34 | 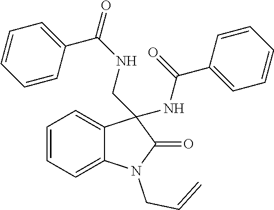 [SMILES: C=CCN1C(=O)C(CNC(=O)c2ccccc2)(NC(=O)c2ccccc2)c2ccccc21] |
| 35 | 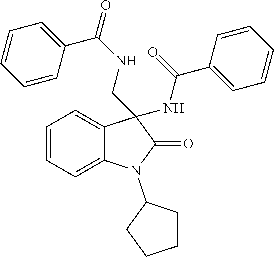 [SMILES: O=C(NCC1(NC(=O)c2ccccc2)C(=O)N(C2CCCC2)c2ccccc21)c1ccccc1] |
| 36 | 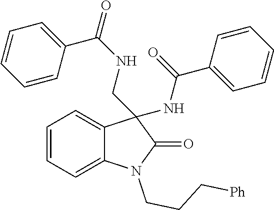 [SMILES: O=C(NCC1(NC(=O)c2ccccc2)C(=O)N(CCCc2ccccc2)c2ccccc21)c1ccccc1] |
| 37 | 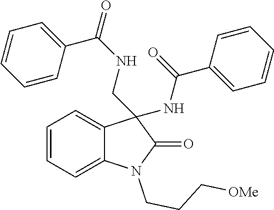 [SMILES: COCCCN1C(=O)C(CNC(=O)c2ccccc2)(NC(=O)c2ccccc2)c2ccccc21] |
| 38 | 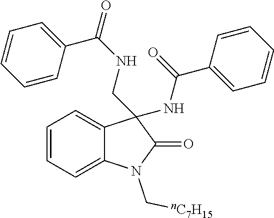 |
| 39 | 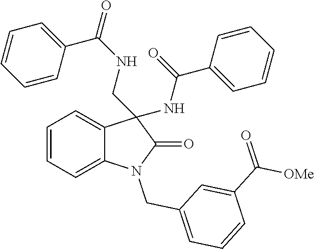 [SMILES: COC(=O)c1cccc(CN2C(=O)C(CNC(=O)c3ccccc3)(NC(=O)c3ccccc3)c3ccccc32)c1] |
| 40 | 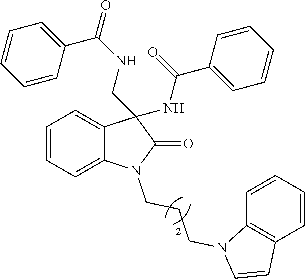 |
| 41 | 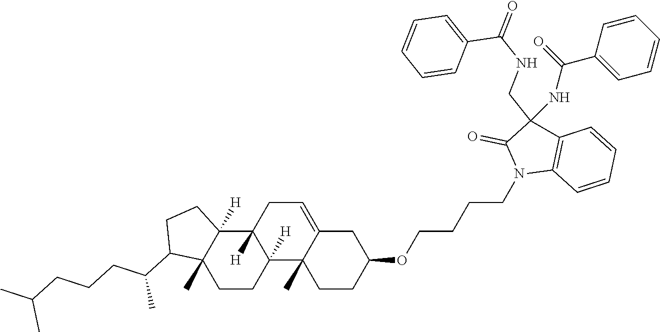 |
| 42 | 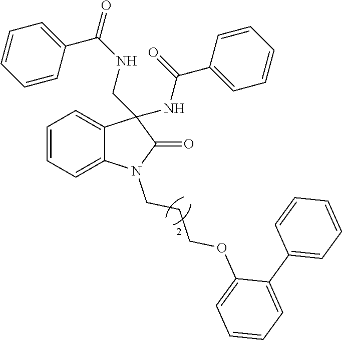 |
| 43 | 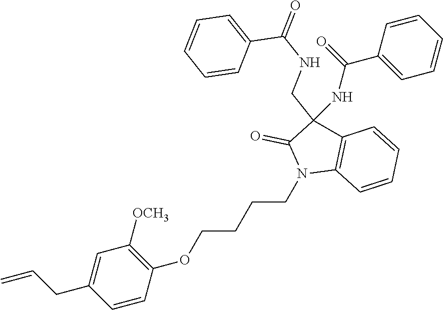 [SMILES: C=CCc1ccc(OCCCCN2C(=O)C(CNC(=O)c3ccccc3)(NC(=O)c3ccccc3)c3ccccc32)c(OC)c1] |
| 44 | 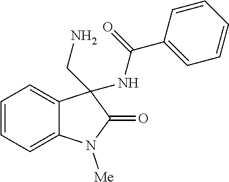 [SMILES: CN1C(=O)C(CN)(NC(=O)c2ccccc2)c2ccccc21] |
| 45 | 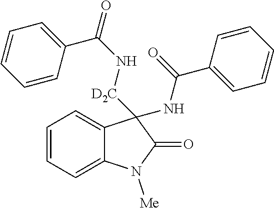 |

In yet another embodiment, present invention provides a process of preparation of the compound of formula (I), isomer, solvate and tautomer form thereof,

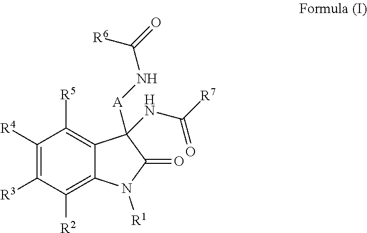

wherein A, R, R¹, R², R³, R⁴, R⁵, R⁶ and R⁷ are as defined above.
comprising the step of:
   i. reacting a compound of formula (II) with a compound of formula (III),

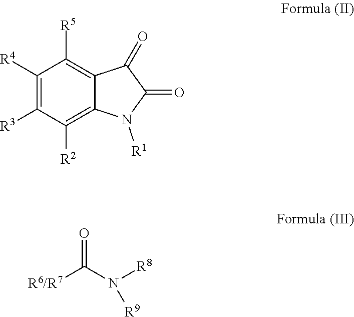

wherein
R¹ is unsubstituted or substituted C₁₋₈ alkyl, unsubstituted or substituted aryl, unsubstituted or substituted alkylaryl, unsubstituted or substituted cycloalkyl, unsubstituted or substituted alkylalkoxy, unsubstituted or substituted alkoxyaryl, unsubstituted or substituted heteroaryl, unsubstituted or substituted alkylheteroaryl, unsubstituted or substituted alkoxyheteroaryl, unsubstituted or substituted ester, unsubstituted or substituted alkyloxy-cycloalkenealkyl, unsubstituted or substituted alkyloxy-aryl, unsubstituted or substituted alkyloxy-heteroaryl, unsubstituted or substituted alkyloxy-arylalkene, or unsubstituted or substituted alkene,
R² is hydrogen, halogen, unsubstituted or substituted C₁₋₆ alkyl, unsubstituted or substituted aryl, or unsubstituted or substituted heteroaryl,
R³ is hydrogen, halogen, unsubstituted or substituted C₁₋₆ alkyl, unsubstituted or substituted aryl, or unsubstituted or substituted heteroaryl,
R⁴ is hydrogen, halogen, unsubstituted or substituted C₁₋₆ alkyl, unsubstituted or substituted alkoxy, unsubstituted or substituted aryl, or unsubstituted or substituted heteroaryl,
R⁵ is hydrogen, halogen, unsubstituted or substituted C₁₋₆ alkyl, unsubstituted or substituted aryl, or unsubstituted or substituted heteroaryl,
R⁶ is unsubstituted or substituted aryl, or unsubstituted or substituted heteroaryl, and
R⁷ is unsubstituted or substituted aryl, or unsubstituted or substituted heteroaryl,
R⁸ is hydrogen, alkoxy, deuterium or deuterated alkoxy,
R⁹ is hydrogen, alkoxy, or deuterated alkoxy.
in a solvent in presence of a base and Fe catalyst to obtain the compound of formula (I).

In yet another embodiment of the present invention, the base is selected from organic or inorganic bases.

In yet another embodiment of the present invention, the base is selected from the group consisting of Lithium tert-butoxide (LiO^{t}Bu), potassium tert-butoxide (KO^{t}Bu), sodium tert-butoxide (NaO^{t}Bu), sodium carbonate (Na₂CO₃), potassium carbonate (K₂CO₃), tripotassium phosphate (K₃PO₄), lithium acetate (LiOAc) and sodium acetate (NaOAc).

In yet another embodiment of the present invention, the solvent is selected from the group consisting of toluene, para-xylene, tert-butyl benzene and 1,4-dioxane.

In yet another embodiment of the present invention, the Fe catalyst can be in one or two or both oxidation state(s) of 2 and/or 3 (Fe²⁺ or Fe³⁺) which provides better product yields.

In yet another embodiment of the present invention, the Fe catalyst is selected from the group consisting of Iron(III) chloride (FeCl₃), Iron(II) chloride (FeCl₂), Iron(III) bromide (FeBr₃), Iron(II) bromide (FeBr₂), Iron(II) acetate [Fe(OAc)₂], Iron(III) acetate [Fe(OAc)₃] and Tris(acetylacetonato)iron(III) [Fe(acac)₃].

In yet another embodiment of the present invention, the process step is carried out at a temperature in a range of 100° C. to 120° C. for the time period in the range of 12 to 24 hrs. In yet another embodiment of the present invention, yield of the compound is in range of 85-95%.

In yet another embodiment of the present invention, the stereogenic 3,3-disubstituted 3-aminooxindole compound of formula (I), or isomer, solvate, and tautomer form thereof of formula (I) is useful in the preparation of bio-relevant C3-aminated oxindoles.

### DETAILED DESCRIPTION OF THE INVENTION

The term, “(C₁₋₈)alkyl”, as used herein, refers to the radical of saturated aliphatic groups, including straight or branched-chain alkyl groups having six or fewer carbon atoms in its backbone, for instance, C₁-C₈ for straight chain and C₃-C₆ for branched chain. As used herein, (C₁₋₆)alkyl refers to an alkyl group having from 1 to 8 carbon atoms. Representative examples of alkyl include, but are not limited to, methyl, ethyl, n-propyl, n-butyl, n-pentyl, n-hexyl, isopropyl, sec-butyl, isobutyl, tert-butyl, isopentyl, 2-methylbutyl and 3-methylbutyl.

Furthermore, unless stated otherwise, the alkyl group can be unsubstituted or substituted with one or more substituents, for example, from one to four substituents, independently selected from the group consisting of halogen, hydroxy, cyano, nitro and amino. Examples of substituted alkyl include, but are not limited to hydroxymethyl, 2-chlorobutyl, trifluoromethyl and aminoethyl.

The term, “halogen” as used herein refers to chlorine, fluorine, and bromine or iodine atom.

The term, “alkoxy” refers to a (C₁₋₁₂)alkyl having an oxygen radical attached thereto. Representative examples of alkoxy groups include, but are not limited to, methoxy, ethoxy, propoxy, isopropoxy, n-butoxy, isobutoxy and tert-butoxy. Furthermore, unless stated otherwise, the alkoxy groups can be unsubstituted or substituted with one or more groups. A substituted alkoxy refers to a (C₁₋₆)alkoxy substituted with one or more groups, particularly one to four groups independently selected from the groups indicated above as the substituents for the alkyl group.

The term “aryl” as used herein refers to monocyclic or bicyclic hydrocarbon groups having 6 to 10 ring carbon atoms, wherein at least one carbocyclic ring is having a π electron system. Examples of (C₆-C₁₀) aryl ring systems include, but are not limited to, phenyl naphthyl and biphenyl. Unless indicated otherwise, aryl group can be unsubstituted or substituted with one or more substituents, for example 1-4 substituents independently selected from the group consisting of halogen, (C₁₋₆)alkyl, hydroxy, cyano, nitro, —COOH, —COO-alkyl, haloalkyl, alkoxy, alkenyl, and amino.

The term “heteroaryl”, as used herein refers to a 5- to 10-membered unsaturated monocyclic or bicyclic aromatic ring system containing 1 to 4 heteroatoms independently selected from the group consisting of oxygen, nitrogen and sulfur. Heteroaryl groups include, but are not limited to, groups such as pyrrolyl pyrazolyl, triazolyl, tetrazolyl, oxazolyl, isoxazolyl, thiazolyl pyridinyl, thiophenyl, benzothiophenyl, benzofuranyl, indolyl azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. Unless stated otherwise, heteroaryl can be unsubstituted or substituted with one or more substituents, for example, substituents independently selected from group consisting of oxo, halogen, haloalkyl, hydroxy, cyano, nitro, amine, (C₁₋₈)alkyl and COOH.

The term “deuterated” refers to a compound which has had some of its normal hydrogen (protium) replaced with the heavy isotope deuterium.

The compounds described herein can be asymmetric (e.g., having one or more stereocenters).

All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiment, compound has(S)-configuration.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of invention can also include all isotopes of atoms occurring in intermediates or final compounds. Isotopes include those atoms having the same atomic number but different mass numbers. For example, isotopes of hydrogen include tritium and deuterium.

The term, “compound,” used herein is meant to include all stereoisomers, geometric isomers, tautomers, and isotopes of structures depicted. Compounds herein identified by name or structure as one particular tautomeric form are intended to include other tautomeric forms unless otherwise specified.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present invention provides a stereogenic 3,3-disubstituted 3-aminooxindole compound of formula (I). The present invention generally relates to a process for generating quaternary stereogenic carbon center. More particularly, the present invention provides an efficient process for the synthesis of stereogenic 3,3-disubstituted 3-aminooxindoles compound of formula (I) by iron-catalyzed reaction of isatins with benzamides.

The present invention relates to a stereogenic 3,3-disubstituted 3-aminooxindole compound of formula (I),

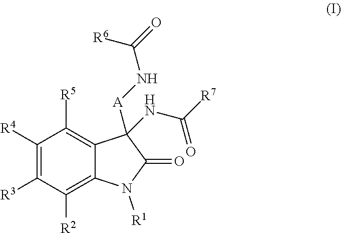

wherein:
A is —CHR—, or —CD₂,
R is hydrogen, or unsubstituted or substituted C₁₋₈ alkyl;
R¹ is unsubstituted or substituted C₁₋₈ alkyl, unsubstituted or substituted aryl, unsubstituted or substituted alkylaryl, unsubstituted or substituted cycloalkyl, unsubstituted or substituted alkylalkoxy, unsubstituted or substituted alkoxyaryl, unsubstituted or substituted heteroaryl, unsubstituted or substituted alkylheteroaryl, unsubstituted or substituted alkoxyheteroaryl, unsubstituted or substituted ester, unsubstituted or substituted alkyloxy-cycloalkenealkyl, unsubstituted or substituted alkyloxy-aryl, unsubstituted or substituted alkyloxy-heteroaryl, unsubstituted or substituted alkyloxy-arylalkene, or unsubstituted or substituted alkene,
R² is hydrogen, halogen, unsubstituted or substituted C₁₋₆alkyl, unsubstituted or substituted aryl, or unsubstituted or substituted heteroaryl,
R³ is hydrogen, halogen, unsubstituted or substituted C₁₋₆alkyl, unsubstituted or substituted aryl, or unsubstituted or substituted heteroaryl,
R⁴ is hydrogen, halogen, unsubstituted or substituted C₁₋₆alkyl, unsubstituted or substituted aryl, or unsubstituted or substituted heteroaryl,
R⁵ is hydrogen, halogen, unsubstituted or substituted C₁₋₆alkyl, unsubstituted or substituted aryl, or unsubstituted or substituted heteroaryl,
R⁶ is unsubstituted or substituted aryl, or unsubstituted or substituted heteroaryl, and
R⁷ is unsubstituted or substituted aryl, or unsubstituted or substituted heteroaryl.

In yet another embodiment of the present invention, the stereogenic 3,3-disubstituted 3-aminooxindole compound of formula (I) is useful in the preparation of bio-relevant C3-aminated oxindoles.
The compound of formula (I) is prepared as given below:

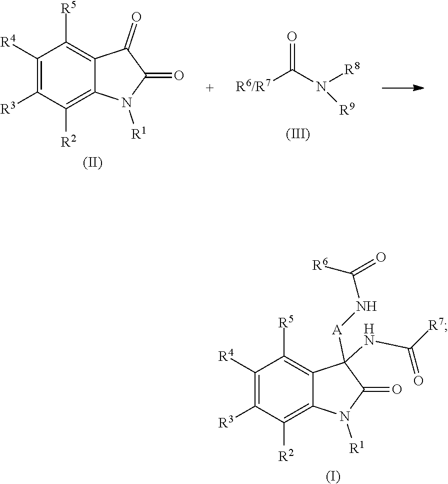

wherein:
A, R¹ to R⁹ are as defined above.

The present invention relates to a process of preparing a stereogenic 3,3-disubstituted 3-aminooxindole compound of formula (I),

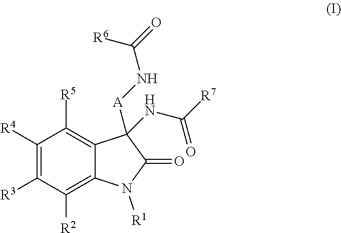

wherein A, R, R¹, R², R³, R⁴, R⁵, R⁶ and R⁷ are as defined above;
comprising the step of:
(i) reacting a compound of formula (II) with a compound of formula (III),

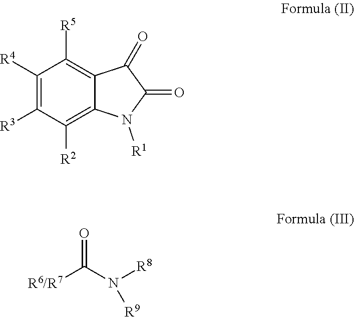

Wherein R¹, R², R³, R⁴, and R⁵, R⁶ and R⁷ are as defined above;
R⁸ is hydrogen, alkoxy, deuterium or deuterated alkoxy,
R⁹ is hydrogen, alkoxy, or deuterated alkoxy.
in a solvent in presence of a base and Fe catalyst to obtain the compound of formula (I).

The preferred base is selected from the group consisting of LiO^{t}Bu, KO^{t}Bu, NaO^{t}Bu, Na₂CO₃, K₂CO₃, K₃PO₄, LiOAc, NaOAc, and the like.

The preferred solvent is selected from the group consisting of toluene, para-xylene, tert-Butyl benzene or 1,4-dioxane.

The preferred catalyst is selected from the group consisting of FeCl₃, FeCl₂, FeBr₃, FeBr₂, Fe(OAc)₂, Fe(OAc)₃ or Fe(acac)₃.

The reaction of compound of formula (II) and (III) is carried out in a temperature in a range of 100° C. to 120° C.

The preferred time of reaction is 12 to 24 hrs.

The yield of compounds is achieved up to 90%.

The process of preparing the stereogenic 3,3-disubstituted 3-aminooxindole compound of formula (I) includes:

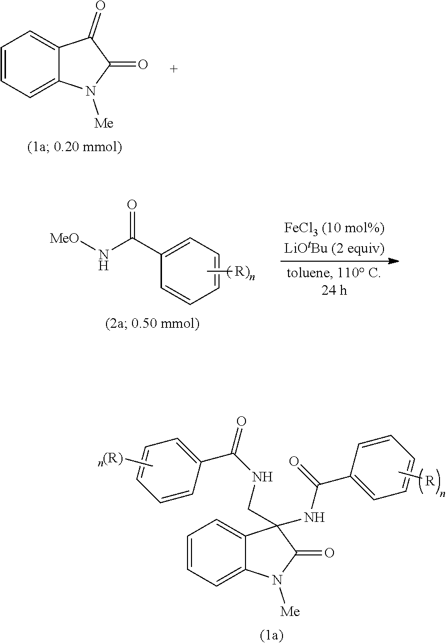

wherein: R is hydrogen, (C₁-C₆)alkyl, alkoxy, halogen, cyano, or haloalkyl; and n is 1, 2 or 3.

In yet another embodiment of the present invention, the process of preparing the stereogenic 3,3-disubstituted 3-aminooxindole compound of formula (I) includes:

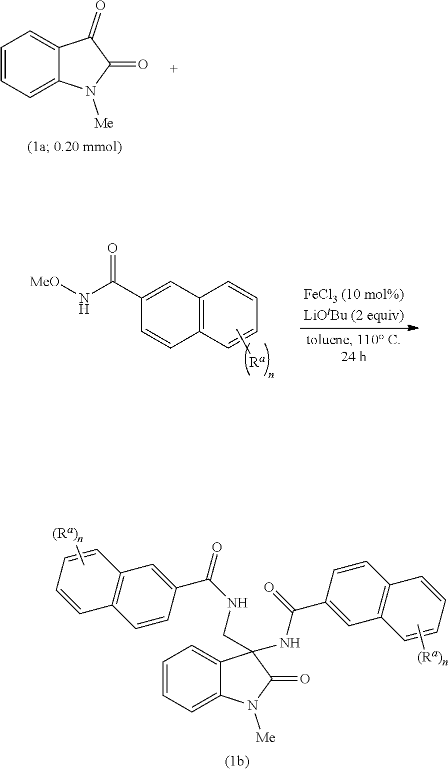

wherein: R is hydrogen, (C₁-C₆)alkyl, alkoxy, halogen, cyano, or haloalkyl; and n is 1, 2 or 3.

In yet another embodiment of the present invention, the process of preparing the stereogenic 3,3-disubstituted 3-aminooxindole compound of formula (I) includes:

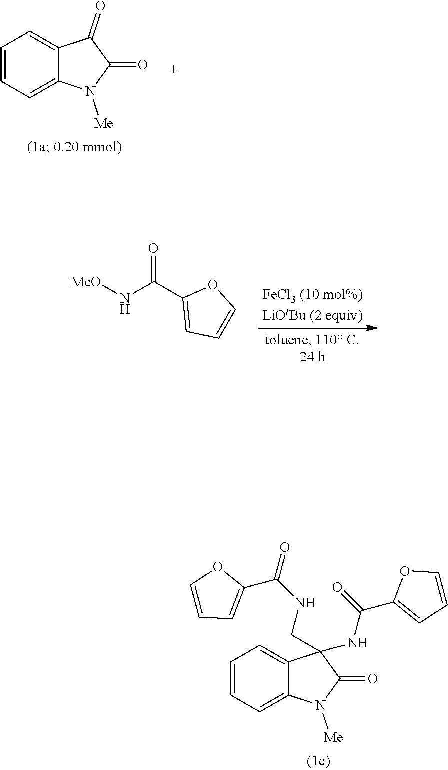

wherein: R^{a} is hydrogen, (C₁-C₆)alkyl, alkoxy, halogen, cyano, or haloalkyl; and n is 1, 2 or 3.

The process of preparing the stereogenic 3,3-disubstituted 3-aminooxindole compound of formula (I) includes:

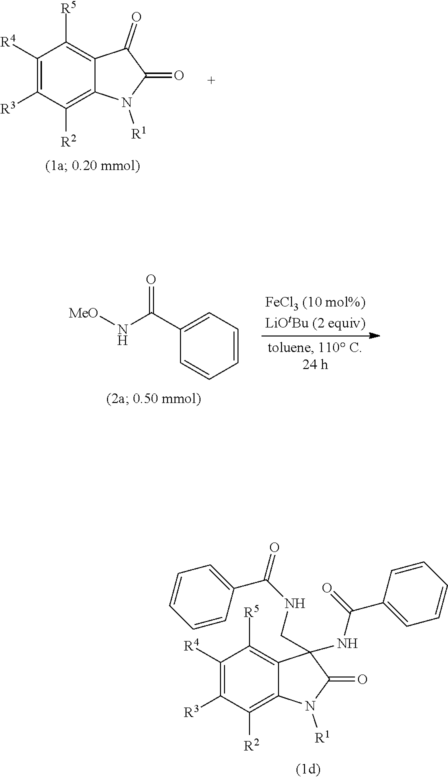

wherein: R¹, R², R³, R⁴, and R⁵ are as defined above.

The present invention provides a process that includes reaction of isatins with N-methoxy benzamides in presence of iron catalyst, base, and solvent at specific temperature and time period to arrive at the 3,3-disubstituted 3-aminooxindole with oxindole scaffold and quaternary stereogenic center at the C3 position.

Iron oxidation state of catalyst can be varied from 2-3 (Fe²⁺ or Fe³⁺) which provides better product yields.

Preferably 5 to 10 mol % of catalyst FeCl₃ is used.

Instead of N-methoxy benzamide (secondary amide), simple primary benzamide is also useful for obtaining said compound.

The preferred base is LiO^{t}Bu, the preferred solvent is toluene, the preferred catalyst is FeCl₃, the preferred temperature is 110° C., and the preferred time of reaction is 24 hrs, but is not limited thereto.

In yet another embodiment of present invention, yield of compounds is achieved up to 90%. Among the range of heterocycles, the presence of a tetrasubstituted quaternary center in oxindoles is advantageous as such motifs are prominent features in various naturally occurring alkaloids and pharmaceutical candidates. Within these structural motifs, 3,3-disubstituted-3-amino-2-oxindole of formula I of the present invention has great synthetic value (as intermediate compound), and is present in cholecystokinin-B receptor antagonists AG-041R, HIV protease inhibitor, anti-malarial agents NITD609, and spirohydantoin DP2 receptor antagonist (Structures shown on page 2).

### EXAMPLES

The following examples are given as a way of illustration only and should not be construed to limit the scope of the present invention.

### Example 1: Preparation of Compound 1 [N-((3-Benzamido-1-methyl-2-oxoindolin-3-yl)methyl)benzamide]

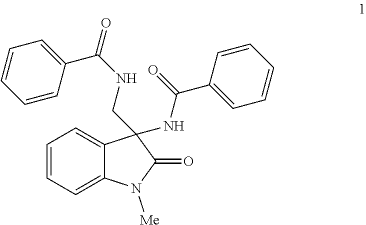 [SMILES: CN1C(=O)C(CNC(=O)c2ccccc2)(NC(=O)c2ccccc2)c2ccccc21]

To a flame-dried screw-cap tube equipped with magnetic stir bar were added 1-methylindoline-2,3-dione (1a; 0.032 g, 0.20 mmol), N-methoxybenzamide (2a; 0.075 g, 0.50 mmol), FeCl₃ (0.0032 g, 0.02 mmol, 10.0 mol %) and LiO^{t}Bu (0.032 g, 0.40 mmol, 2 equiv) inside the glove box. To the above mixture in the tube was added toluene (1.0 mL). The resultant reaction mixture in the tube was immersed in a preheated oil bath at 110° C. and stirred for 24 h. At ambient temperature, the reaction mixture was quenched with distilled H₂O (10.0 mL) and the crude product was extracted with EtOAc (15 mL×3). The combined organic extract was dried over Na₂SO₄ and the volatiles were evaporated in vacuo. The remaining residue was purified by column chromatography on silica gel (petroleum ether/EtOAc: 1/2) to yield the title compound 1.

¹H-NMR (500 MHz, CDCl₃): δ=9.25 (br s, 1H, NH), 7.86 (d, J=7.1 Hz, 2H, Ar—H), 7.79 (d, J=7.1 Hz, 2H, Ar—H), 7.68 (br s, 1H, NH), 7.50-7.47 (m, 1H, Ar—H), 7.42 (t, J=7.7 Hz, 3H, Ar—H), 7.38-7.32 (m, 1H, Ar—H), 7.28-7.20 (m, 3H, Ar—H), 7.04 (t, J=7.3 Hz, 1H, Ar—H), 6.90 (d, J=7.7 Hz, 1H, Ar—H), 4.23 (dd, J=14.5, 7.8 Hz, 1H, CH₂), 3.41 (dd, J=14.5, 5.0 Hz, 1H, CH₂), 3.25 (s, 3H, CH₃). ¹³C{¹H}-NMR (125 MHz, CDCl₃): δ=175.4 (CO), 170.9 (CO), 166.3 (CO), 143.3 (C_{q}), 132.9 (C_{q}), 132.5 (C_{q}), 132.3 (CH), 132.0 (CH), 129.6 (CH), 128.7 (2C, CH), 128.6 (2C, CH), 128.3 (C_{q}), 127.7 (2C, CH), 127.5 (2C, CH), 123.2 (CH), 122.8 (CH), 108.7 (CH), 63.4 (C_{q}), 45.7 (CH₂), 26.7 (CH₃). HRMS (ESI): m/z Calcd for C₂₄H₂₁N₃O₃+H⁺ [M+H]⁺400.1656; Found 400.1652.

### Examples 2-45

The examples 2-45 were prepared by following the experimental procedure as given in the example 1 with appropriate staring materials and non-critical variations.

| Ex. | Compound | |
|---|---|---|
| No | No. | Characterization data |
| 2 | 2 | ¹H-NMR (500 MHz, CDCl₃): δ = 9.12 (br s, 1H, NH), 7.77 (d, J = 8.2 Hz, |
| | | 2H, Ar—H), 7.68 (d, J = 7.7 Hz, 2H, Ar—H), 7.48 (br s, 1H, NH), 7.36-7.32 |
| | | (m, 1H, Ar—H), 7.29 (d, J = 7.2 Hz, 1H, Ar—H), 7.22 (d, J = 8.0 Hz, 2H, |
| | | Ar—H), 7.08-7.01 (m, 3H, Ar—H), 6.90 (J = 7.7 Hz, 1H, Ar—H), 4.23 (dd, |
| | | J = 14.5, 7.9 Hz, 1H, CH₂), 3.43 (dd, J = 14.5, 5.0 Hz, 1H, CH₂), 3.29 (s, |
| | | 3H, CH₃), 2.38 (s, 3H, CH₃), 2.31 (s, 3H, CH₃). ¹³C{¹H}-NMR (125 MHz, |
| | | CDCl₃): δ = 175.5 (CO), 170.8 (CO), 166.3 (CO), 143.4 (C_{q}), 142.8 (C_{q}), |
| | | 142.4 (C_{q}), 130.2 (C_{q}), 129.8 (C_{q}), 129.6 (CH), 129.4 (2C, CH), 129.3 |
| | | (2C, CH), 128.5 (C_{q}), 127.8 (2C, CH), 127.5 (2C, CH), 123.2 (CH), 122.8 |
| | | (CH), 108.6 (CH), 63.4 (C_{q}), 45.6 (CH₂), 26.7 (CH₃), 21.6 (CH₃), 21.5 |
| | | (CH₃). HRMS (ESI): m/z Calcd for C₂₆H₂₅N₃O₃ + H⁺ [M + H]⁺ 428.1969; |
| | | Found 428.1966. |
| 3 | 3 | ¹H-NMR (400 MHz, CDCl₃): δ = 9.22 (br s, 1H, NH), 7.81 (d, J = 7.6 Hz, |
| | | 2H, Ar—H), 7.71-7.66 (m, 1H, NH; 2H, Ar—H), 7.34 (t, J = 7.7 Hz, 1H, |
| | | Ar—H), 7.29 (d, J = 7.2 Hz, 1H, Ar—H), 7.23 (d, J = 7.6 Hz, 2H, Ar—H), |
| | | 7.05 (t, J = 7.5 Hz, 1H, Ar—H), 6.98 (d J = 7.6 Hz, 2H, Ar—H), 6.91 (d |
| | | J = 7.7 Hz, 1H, Ar—H), 4.25 (dd, J = 14.3, 7.6 Hz, 1H, CH₂), 3.40 (dd, J = |
| | | 14.3, 4.3 Hz, 1H, CH₂), 3.27 (s, 3H, CH₃), 2.63 (t, J = 7.6 Hz, 2H, CH₂), |
| | | 2.53 (t, J = 7.6 Hz, 2H, CH₂), 1.61-1.50 (m, 4H, CH₂), 1.37-1.30 (m, 4H, |
| | | CH₂), 0.95-0.90 (m, 6H, CH₃). ¹³C{¹H}-NMR (100 MHz, CDCl₃): δ = |
| | | 175.6 (CO), 170.8 (CO), 166.3 (CO), 147.5 (C_{q}), 147.4 (C_{q}), 143.3 (C_{q}), |
| | | 130.1 (C_{q}), 130.0 (2C, C_{q}), 129.6 (CH), 128.7 (3C, CH), 128.6 (CH), |
| | | 127.8 (2C, CH), 127.5 (2C, CH), 123.2 (CH), 122.8 (CH), 108.6 (CH), |
| | | 63.5 (C_{q}), 45.8 (CH₂), 35.7 (2C, CH₂), 33.5 (2C, CH₂), 26.7 (CH₃), 22.5 |
| | | (2C, CH₂), 14.1 (2C, CH₃). HRMS (ESI): m/z Calcd for C₃₂H₃₇N₃O₃ + H⁺ |
| | | [M + H]⁺ 512.2916; Found 512.2914. |
| 4 | 4 | ¹H-NMR (500 MHz, CDCl₃): δ = 9.14 (br s, 1H, NH), 7.84 (d, J = 8.7 Hz, |
| | | 2H, Ar—H), 7.79 (d, J = 8.7 Hz, 2H, Ar—H), 7.36-7.29 (m, 1H, NH; 2H, |
| | | Ar—H), 7.06 (t, J = 7.5 Hz, 1H, Ar—H), 6.91 (t, J = 8.0 Hz, 3H, Ar—H), |
| | | 7.78 (d, J = 8.7 Hz, 2H, Ar—H), 4.23-4.17 (m, 1H, CH₂), 3.83 (s, 3H, |
| | | OCH₃), 3.79 (s, 3H, OCH₃), 3.45 (dd, J = 14.6, 4.9 Hz, 1H, CH₂), 3.30 (s, |
| | | 3H, CH₃). ¹³C{¹H}-NMR (125 MHz, CDCl₃): δ = 175.5 (CO), 170.4 |
| | | (CO), 165.9 (CO), 162.8 (C_{q}), 162.5 (C_{q}), 143.3 (C_{q}), 129.6 (2C, CH), |
| | | 129.4 (2C, CH), 128.5 (C_{q}), 125.3 (C_{q}), 125.1 (C_{q}), 123.1 (CH), 122.8 |
| | | (CH), 113.9 (2C, CH), 113.8 (2C, CH), 108.7 (2C, CH), 63.4 (C_{q}), 55.6 |
| | | (OCH₃), 55.5 (OCH₃), 45.6 (CH₂), 26.7 (CH₃). HRMS (ESI): m/z Calcd |
| | | for C₂₆H₂₅N₃O₅ + H⁺ [M + H]⁺ 460.1867; Found 460.1867. |
| 5 | 5 | ¹H-NMR (500 MHz, CDCl₃): δ = 9.35 (br s, 1H, NH), 7.92-7.88 (m, 3H, |
| | | Ar—H), 7.81-7.78 (m, 2H, Ar—H), 7.38-7.34 (m, 1H, Ar—H), 7.30 (d, J = |
| | | 7.3 Hz, 1H, Ar—H), 7.14-7.06 (m, 1H, NH; 2H, Ar—H), 6.93 (d, J = 7.7 |
| | | Hz, 1H, Ar—H), 6.79 (t, J = 8.5 Hz, 2H, Ar—H), 4.34 (dd, J = 14.6, 7.7 Hz, |
| | | 1H, CH₂), 3.32 (dd, J = 14.6, 5.1 Hz, 1H, CH₂), 3.25 (s, 3H, CH₃). |
| | | ¹³C{¹H}-NMR (125 MHz, CDCl₃): δ = 175.5 (CO), 169.9 (CO), 165.3 (d, |
| | | ¹J_{C—F} = 252.5 Hz, C_{q}), 165.2 (d, ¹J_{C—F} = 253.3 Hz, C_{q}), 165.1 (CO), 143.2 |
| | | (C_{q}), 130.1 (d, ³J_{C—F} = 16.7 Hz, 2C, CH), 130.0 (d, ³J_{C—F} = 16.7 Hz, 2C, |
| | | CH), 129.8 (CH), 128.9 (d, ⁴J_{C—F} = 3.0 Hz, C_{q}), 128.7 (d, ⁴J_{C—F} = 3.0 Hz, |
| | | C_{q}), 128.2 (C_{q}), 123.4 (CH), 122.7 (CH), 115.8 (d, ²J_{C—F} = 21.3 Hz, 2C, |
| | | CH), 115.7 (d, ²J_{C—F} = 22.1 Hz, 2C, CH), 108.8 (CH), 63.6 (C_{q}), 45.9 |
| | | (CH₂), 26.7 (CH₃). ¹⁹F-NMR (376 MHz, CDCl₃): δ = −106.9 (s), −107.3 |
| | | (s). HRMS (ESI): m/z Calcd for C₂₄H₁₉F₂N₃O₃ + H⁺ [M + H]⁺ 436.1467; |
| | | Found 436.1464. |
| 6 | 6 | ¹H-NMR (500 MHz, CDCl₃): δ = 9.41 (br s, 1H, NH), 8.04 (br s, 1H, NH), |
| | | 7.85 (d, J = 8.5 Hz, 2H, Ar—H), 7.67 (d, J = 8.6 Hz, 2H, Ar—H), 7.45 (d, |
| | | J = 8.5 Hz, 2H, Ar—H), 7.38 (t, J = 7.3 Hz, 1H, Ar—H), 7.32 (d, J = 7.0 Hz, |
| | | 1H, Ar—H), 7.11 (t, J = 7.5 Hz, 1H, Ar—H), 6.98-6.93 (m, 3H, Ar—H), 4.38 |
| | | (dd, J = 14.6, 7.7 Hz, 1H, CH₂), 3.31-3.26 (m, 1H, CH₂; 3H, CH₃). |
| | | ¹³C{¹H}-NMR (125 MHz, CDCl₃): δ = 175.5 (CO), 170.0 (CO), 165.2 |
| | | (CO), 143.1 (C_{q}), 138.7 (C_{q}), 138.6 (C_{q}), 130.8 (C_{q}), 130.7 (C_{q}), 129.9 |
| | | (CH), 129.2 (2C, CH), 129.1 (2C, CH), 128.9 (2C, CH), 128.8 (2C, CH), |
| | | 128.1 (C_{q}), 123.5 (CH), 122.8 (CH), 108.8 (CH), 63.7 (C_{q}), 45.9 (CH₂), |
| | | 26.7 (CH₃). HRMS (ESI): m/z Calcd for C₂₄H₁₉C₁₂N₃O₃ + H⁺ [M + H]⁺ |
| | | 468.0884; Found 468.0882. |
| 7 | 7 | ¹H-NMR (400 MHz, CDCl₃): δ = 9.49 (br s, 1H, NH), 8.20 (br s, 1H, NH), |
| | | 7.79 (d, J = 7.1 Hz, 2H, Ar—H), 7.63 (d, J = 7.2 Hz, 2H, Ar—H), 7.56 (d, |
| | | J = 7.2 Hz, 2H, Ar—H), 7.40-7.33 (m, 2H, Ar—H), 7.12 (t, J = 7.3 Hz, 1H, |
| | | Ar—H), 7.02 (d, J = 7.5 Hz, 2H, Ar—H), 6.94 (d, J = 7.7 Hz, 1H, Ar—H), |
| | | 4.41 (dd, J = 14.4, 7.2 Hz, 1H, CH₂), 3.28-3.24 (m, 1H, CH₂; 3H, CH₃). |
| | | ¹³C{¹H}-NMR (100 MHz, CDCl₃): δ = 175.7 (CO), 170.0 (CO), 165.3 |
| | | (CO), 142.9 (C_{q}), 132.3 (2C, CH), 131.7 (2C, CH), 131.2 (C_{q}), 131.0 (C_{q}), |
| | | 129.9 (CH), 129.3 (2C, CH), 129.0 (2C, CH), 128.1 (C_{q}), 127.4 (C_{q}), |
| | | 127.2 (C_{q}), 123.6 (CH), 122.8 (CH), 108.8 (CH), 63.8 (C_{q}), 46.1 (CH₂), |
| | | 26.7 (CH₃). HRMS (ESI): m/z Calcd for C₂₄H₁₉Br₂N₃O₃ + H⁺ [M + H]⁺ |
| | | 557.9851; Found 557.9852. |
| 8 | 8 | ¹H-NMR (400 MHz, CDCl₃): δ = 9.57 (br s, 1H, NH), 8.38 (br s, 1H, NH), |
| | | 7.86 (d, J = 7.9 Hz, 2H, Ar—H), 7.66 (d, J = 7.7 Hz, 2H, Ar—H), 7.40-7.34 |
| | | (m, 4H, Ar—H), 7.19-7.10 (m, 3H, Ar—H), 6.95 (d, J = 7.7 Hz, 1H, Ar—H), |
| | | 4.44 (dd, J = 14.3, 7.2 Hz, 1H, CH₂), 3.31-3.23 (m, 1H, CH₂; 3H, CH₃). |
| | | ¹³C{¹H}-NMR (100 MHz, CDCl₃): δ = 175.6 (CO), 170.2 (CO), 165.5 (CO), |
| | | 142.9 (C_{q}), 138.3 (2C, CH), 137.7 (2C, CH), 131.7 (C_{q}), 131.5 (C_{q}), |
| | | 129.9 (CH), 129.2 (2C, CH), 128.9 (2C, CH), 128.1 (C_{q}), 123.7 (CH), |
| | | 122.8 (CH), 108.8 (CH), 100.1 (C_{q}), 99.9 (C_{q}), 63.9 (C_{q}), 46.1 (CH₂), 26.7 |
| | | (CH₃). HRMS (ESI): m/z Calcd for C₂₄H₁₉₁₂N₃O₃ + H⁺ [M + H]⁺ |
| | | 651.9606; Found 651.9604. |
| 9 | 9 | ¹H-NMR (500 MHz, CDCl₃): δ = 9.13 (br s, 1H, NH), 7.89-7.86 (m, 4H, |
| | | Ar—H), 7.66 (d, J = 7.7 Hz, 2H, Ar—H), 7.58 (d, J = 7.6 Hz, 2H, Ar—H), |
| | | 7.46 (br s, 1H, NH), 7.32 (t, J = 7.7 Hz, 1H, Ar—H), 7.22-7.19 (m, 1H, Ar— |
| | | H), 7.03 (vt, J = 7.4 Hz, 1H, Ar—H), 6.89 (d, J = 7.7 Hz, 1H, Ar—H), 4.26 |
| | | (dd, J = 14.6, 8.1 Hz, 1H, CH₂), 3.34-3.30 (m, 1H, CH₂), 3.23 (s, 3H, |
| | | CH₃). ¹³C{¹H}-NMR (125 MHz, CDCl₃): δ = 174.7 (CO), 169.4 (CO), |
| | | 164.4 (CO), 143.3 (C_{q}), 136.7 (CN), 136.2 (CN), 132.7 (2C, CH), 132.6 |
| | | (2C, CH), 130.2 (CH), 128.3 (2C, CH), 128.2 (2C, CH), 127.4 (C_{q}), 123.5 |
| | | (CH), 122.7 (CH), 118.1 (C_{q}), 117.8 (C_{q}), 116.2 (C_{q}), 115.8 (C_{q}), 109.1 |
| | | (CH), 63.5 (C_{q}), 45.7 (CH₂), 26.9 (CH₃). HRMS (ESI): m/z Calcd for |
| | | C₂₆H₁₉N₅O₃ + H⁺ [M + H]⁺ 450.1561; Found 450.1553. |
| 10 | 10 | ¹H-NMR (500 MHz, CDCl₃): δ = 9.00 (br s, 1H, NH), 7.67-7.65 (m, 3H, |
| | | Ar—H), 7.64 (br s, 1H, NH), 7.36-7.31 (m, 1H, Ar—H), 7.30-7.23 (m, 6H, |
| | | Ar—H), 7.05 (t, J = 7.6 Hz, 1H, Ar—H), 6.90 (d, J = 7.7 Hz, 1H, Ar—H), |
| | | 4.22 (dd, J = 14.6, 8.2 Hz, 1H, CH₂), 3.45 (dd, J = 14.5, 4.9 Hz, 1H, CH₂), |
| | | 3.30 (s, 3H, CH₃), 2.36 (s, 6H, CH₃). ¹³C{¹H}-NMR (125 MHz, CDCl₃): |
| | | δ = 175.3 (CO), 171.0 (CO), 166.5 (CO), 143.5 (C_{q}), 138.8 (C_{q}), 138.4 |
| | | (C_{q}), 133.2 (CH), 133.1 (C_{q}), 132.7 (CH), 132.5 (C_{q}), 129.7 (CH), 128.8 |
| | | (CH), 128.6 (CH), 128.5 (CH), 128.3 (C_{q}), 128.2 (CH), 124.6 (CH), 124.5 |
| | | (CH), 123.1 (CH), 122.8 (CH), 108.7 (CH), 63.2 (C_{q}), 45.4 (CH₂), 26.8 |
| | | (CH₃), 21.5 (CH₃), 21.4 (CH₃). HRMS (ESI): m/z Calcd for C₂₆H₂₅N₃O₃ + |
| | | H⁺ [M + H]⁺ 428.1969; Found 428.1967. |
| 11 | 11 | ¹H-NMR (400 MHz, CDCl₃): δ = 9.25 (br s, 1H, NH), 7.22 (br s, 1H, NH), |
| | | 7.65 (d, J = 7.7 Hz, 1H, Ar—H), 7.59-7.51 (m, 3H, Ar—H), 7.45-7.34 (m, |
| | | 2H, Ar—H), 7.29 (d, J = 7.2 Hz, 1H, Ar—H), 7.22-7.18 (m, 1H, Ar—H), |
| | | 7.14-7.06 (m, 3H, Ar—H), 6.93 (d, J = 7.7 Hz, 1H, Ar—H), 4.30 (dd, J = |
| | | 14.6, 7.8 Hz, 1H, CH₂), 3.36 (dd, J = 14.6, 5.0 Hz, 1H, CH₂), 3.26 (s, 3H, |
| | | CH₃). ¹³C{¹H}-NMR (100 MHz, CDCl₃): δ = 175.2 (CO), 169.7 (d, ⁴J_{C—F} = |
| | | 2.3 Hz, CO), 165.1 (d, ⁴J_{C—F} = 2.3 Hz, CO), 162.9 (d, ¹J_{C—F} = 247.2 Hz, |
| | | C_{q}), 162.8 (d, ¹J_{C—F} = 248.7 Hz, C_{q}), 143.2 (C_{q}), 135.0 (d, ³J_{C—F} = 6.9 Hz, |
| | | C_{q}), 134.8 (d, ³J_{C—F} = 7.6 Hz, C_{q}), 130.4 (d, ³J_{C—F} = 7.6 Hz, 2C, CH), 129.9 |
| | | (CH), 127.9 (C_{q}), 123.4 (CH), 123.1 (d, ⁴J_{C—F} = 2.3 Hz, CH), 122.8 (d, |
| | | ⁴J_{C—F} = 3.0 Hz, CH), 122.7 (CH), 119.5 (d, ²J_{C—F} = 21.4 Hz, CH), 119.1 |
| | | (d, ²J_{C—F} = 21.4 Hz, CH), 115.1 (d, ²J_{C—F} = 23.6 Hz, CH), 115.0 (d, ²J_{C—F} = |
| | | 22.8 Hz, CH), 108.8 (CH), 63.5 (C_{q}), 45.8 (CH₂), 26.8 (CH₃). ¹⁹F-NMR |
| | | (376 MHz, CDCl₃): δ = −111.3 (s), −111.8 (s). HRMS (ESI): m/z Calcd |
| | | for C₂₄H₁₉F₂N₃O₃ + H⁺ [M + H]⁺ 436.1467; Found 436.1454. |
| 12 | 12 | ¹H-NMR (500 MHz, CDCl₃): δ = 9.33 (br s, 1H, NH), 7.87-7.82 (m, 1H, |
| | | NH; 2H, Ar—H), 7.74 (d, J = 7.7 Hz, 1H, Ar—H), 7.70 (d, J = 7.8 Hz, 1H, |
| | | Ar—H), 7.48 (d, J = 8.0 Hz, 1H, Ar—H), 7.41-7.35 (m, 3H, Ar—H), 7.30 (d, |
| | | J = 7.2 Hz, 1H, Ar—H), 7.09 (t, J = 7.3 Hz, 2H, Ar—H), 6.93 (d, J = 7.7 |
| | | Hz, 1H, Ar—H), 4.34 (dd, J = 14.6, 7.7 Hz, 1H, CH₂), 3.34 (dd, J = 14.6, |
| | | 5.0 Hz, 1H, CH₂), 3.25 (CH₃). ¹³C{¹H}-NMR (125 MHz, CDCl₃): δ = |
| | | 175.2 (CO), 169.6 (CO), 165.1 (CO), 143.2 (C_{q}), 135.1 (C_{q}), 134.9 (C_{q}), |
| | | 134.3 (C_{q}), 134.2 (C_{q}), 130.0 (CH), 129.9 (2C, CH), 128.3 (CH), 128.1 |
| | | (2C, CH), 127.9 (C_{q}), 125.6 (2C, CH), 125.4 (CH), 123.5 (CH), 122.8 |
| | | (CH), 108.8 (CH), 63.5 (C_{q}), 45.7 (CH₂), 26.8 (CH₃). HRMS (ESI): m/z |
| | | Calcd for C₂₄H₁₉C₁₂N₃O₃ + H⁺ [M + H]⁺ 468.1288; Found 468.1285. |
| 13 | 13 | ¹H-NMR (400 MHz, DMSO-d₆): δ = 9.68 (br s, 1H, NH), 9.18 (br s, 1H, |
| | | NH), 8.18 (s, 1H, Ar—H), 8.12 (d, J = 7.8 Hz, 1H, Ar—H), 8.06 (s, 2H, |
| | | Ar—H), 7.97-7.92 (m, 2H, Ar—H), 7.79-7.72 (m, 2H, Ar—H), 7.30-7.27 (m, |
| | | 2H, Ar—H), 7.03 (d, J = 7.8 Hz, 1H, Ar—H), 6.96 (t, J = 7.5 Hz, 1H, Ar— |
| | | H), 3.99 (dd, J = 13.8, 6.7 Hz, 1H, CH₂), 3.67 (dd, J = 13.8, 5.8 Hz, 1H, |
| | | CH₂), 3.21 (s, 3H, CH₃). ¹³C{¹H}-NMR (125 MHz, DMSO-d₆): δ = 174.0 |
| | | (CO), 167.3 (CO), 163.8 (CO), 143.7 (C_{q}), 134.7 (C_{q}), 133.8 (C_{q}), 131.5 |
| | | (2C, CH), 130.1 (CH), 129.9 (CH), 129.4 (q, ²J_{C—F} = 32.0 Hz, C_{q}), 129.2 |
| | | (q, ²J_{C—F} = 32.0 Hz, C_{q}), 128.9 (CH), 128.5 (q, ³J_{C—F} = 3.0 Hz, CH), 128.3 |
| | | (q, ³J_{C—F} = 3.0 Hz, CH), 128.3 (C_{q}), 124.2 (q, ³J_{C—F} = 3.8 Hz, CH), 123.7 |
| | | (q ³J_{C—F} = 3.8 Hz, CH), 123.9 (q, ¹J_{C—F} = 272.2 Hz, 2C, CF₃), 123.0 (CH), |
| | | 122.1 (CH), 108.2 (CH), 62.9 (C_{q}), 44.7 (CH₂), 26.5 (CH₃). ¹⁹F-NMR |
| | | (376 MHz, DMSO-d₆): δ = −56.5 (s), −56.6 (s). HRMS (ESI): m/z Calcd |
| | | for C₂₆H₁₉F₆N₃O₃ + H⁺ [M + H]⁺ 536.1403; Found 536.1404. |
| 14 | 14 | ¹H-NMR (400 MHz, CDCl₃): δ = 8.50 (br s, 1H, NH), 8.24 (t, J = 9.8 Hz, |
| | | 2H, Ar—H), 7.93 (d, J = 8.1 Hz, 1H, Ar—H), 7.87 (d, J = 9.3 Hz, 2H, Ar— |
| | | H), 7.80 (d, J = 8.0 Hz, 1H, Ar—H), 7.76 (d, J = 7.0 Hz, 1H, Ar—H), 7.71 |
| | | (d, J = 7.0 Hz, 1H, Ar—H), 7.50-7.33 (m, 1H, NH; 7H, Ar—H), 7.12-7.06 |
| | | (m, 2H, Ar—H), 6.95 (d, J = 7.7 Hz, 1H, Ar—H), 4.43 (dd, J = 14.3, 8.6 Hz, |
| | | 1H, CH₂), 3.39 (dd, J = 14.5, 3.6 Hz, 1H, CH₂), 3.34 (s, 3H, CH₃). |
| | | ¹³C{¹H}-NMR (100 MHz, CDCl₃): δ = 175.2 (CO), 172.4 (CO), 169.1 |
| | | (CO), 143.7 (C_{q}), 133.9 (C_{q}), 133.8 (C_{q}), 132.9 (C_{q}), 132.7 (C_{q}), 131.7 |
| | | (2C, CH), 131.1 (CH), 130.5 (C_{q}), 130.2 (C_{q}), 129.7 (CH), 128.5 (CH), |
| | | 128.3 (CH), 128.2 (C_{q}), 127.5 (CH), 127.3 (CH), 126.7 (CH), 126.3 (CH), |
| | | 125.8 (CH), 125.7 (CH), 125.6 (CH), 125.3 (CH), 124.9 (CH), 123.2 |
| | | (CH), 122.7 (CH), 108.9 (CH), 63.0 (C_{q}), 45.0 (CH₂), 26.9 (CH₃). HRMS |
| | | (ESI): m/z Calcd for C₃₂H₂₅N₃O₃ + H⁺ [M + H]⁺ 500.1969; Found |
| | | 500.1916. |
| 15 | 15 | ¹H-NMR (400 MHz, CDCl₃): δ = 9.23 (br s, 1H, NH), 7.87-7.81 (m, 4H, |
| | | Ar—H), 7.66 (br s, 1H, NH), 7.51-7.39 (m, 4H, Ar—H), 7.31-7.27 (m, 2H, |
| | | Ar—H), 7.03-6.98 (m, 2H, Ar—H), 6.84-6.81 (m, 1H, Ar—H), 4.14 (dd, J = |
| | | 14.5, 7.7 Hz, 1H, CH₂), 3.83-3.71 (m, 2H, CH₂), 3.44 (dd, J = 14.5, 4.9 |
| | | Hz, 1H, CH₂), 1.30 (t, J = 7.1 Hz, 3H, CH₃). ¹³C{¹H}-NMR (100 MHz, |
| | | CDCl₃): δ = 174.8 (CO), 170.9 (CO), 165.5 (CO), 159.4 (d, ¹J_{C—F} = 241.8 |
| | | Hz, C_{q}), 138.3 (C_{q}), 132.9 (C_{q}), 132.4 (C_{q}), 132.3 (CH), 132.1 (CH), 130.2 |
| | | (d, ³J_{C—F} = 7.6 Hz, C_{q}), 128.8 (2C, CH), 128.7 (2C, CH), 127.7 (2C, CH), |
| | | 127.5 (2C, CH), 115.7 (d, ²J_{C—F} = 23.6 Hz, CH), 111.2 (d, ²J_{C—F} = 25.2 Hz, |
| | | CH), 109.4 (d, ³J_{C—F} = 7.6 Hz, CH), 63.5 (C), 45.6 (CH₂), 35.4 (CH₂), |
| | | 12.4 (CH₃). ¹⁹F-NMR (376 MHz, CDCl₃): δ = −119.9 (s). HRMS (ESI): |
| | | m/z Calcd for C₂₅H₂₂FN₃O₃ + H⁺ [M + H]⁺ 432.1718; Found 432.1723. |
| 16 | 16 | 1H-NMR (400 MHz, CDCl₃): δ = 9.17 (br s, 1H, NH), 7.35 (t, J = 7.7 Hz, |
| | | 1H, Ar—H), 7.31 (d, J = 7.2 Hz, 1H, Ar—H), 7.20 (br s, 1H, NH), 7.15 (s, |
| | | 2H, Ar—H), 7.11 (s, 2H, Ar—H), 7.09-7.04 (m, 1H, Ar—H), 6.92 (d, J = 7.7 |
| | | Hz, 1H, Ar—H), 4.19 (dd, J = 14.5, 8.0 Hz, 1H, CH₂), 3.88 (s, 9H, OCH₃), |
| | | 3.86 (s, 3H, OCH₃), 3.85 (s, 6H, OCH₃), 3.47 (dd, J = 14.5, 5.0 Hz, 1H, |
| | | CH₂), 3.30 (s, 3H, CH₃). ¹³C{¹H}-NMR (100 MHz, CDCl₃): δ = 175.1 |
| | | (CO), 170.8 (CO), 165.7 (CO), 153.4 (2C, C_{q}), 153.1 (2C, C_{q}), 143.5 (C_{q}), |
| | | 141.9 (C_{q}), 141.2 (C_{q}), 129.8 (CH), 128.3 (C_{q}), 128.1 (C_{q}), 127.7 (C_{q}), |
| | | 123.2 (CH), 122.8 (CH), 108.7 (CH), 104.9 (2C, CH), 104.8 (2C, CH), |
| | | 63.5 (C_{q}), 61.1 (2C, OCH₃), 56.4 (2C, OCH₃), 56.3 (2C, OCH₃), 45.7 |
| | | (CH₂), 26.9 (CH₃). HRMS (ESI): m/z Calcd for C₃₀H₃₃N₃O₉ + H⁺ [M + |
| | | H]⁺ 580.2290; Found 580.2283. |
| 17 | 17 | ¹H-NMR (500 MHz, DMSO-d₆): δ = 9.47 (br s, 1H, NH), 8.99 (br s, 1H, |
| | | NH), 7.83-7.80 (m, 2H, Ar—H), 7.78-7.77 (m, 1H, Ar—H), 7.74-7.73 (m, |
| | | 1H, Ar—H), 7.29 (t, J = 7.6 Hz, 1H, Ar—H), 7.23-7.16 (m, 3H, Ar—H), 7.03 |
| | | (t, J = 7.9 Hz, 1H, Ar—H), 6.98 (vt, J = 7.4 Hz, 1H, Ar—H), 3.84 (dd, J = |
| | | 14.0, 6.1 Hz, 1H, CH₂), 3.52 (dd, J = 14.0, 6.1 Hz, 1H, CH₂), 3.18 (s, 3H, |
| | | CH₃). ¹³C{¹H}-NMR (125 MHz, DMSO-d₆): δ = 173.9 (CO), 163.5 (CO), |
| | | 159.9 (CO), 143.5 (C_{q}), 138.4 (C_{q}), 138.2 (C_{q}), 131.8 (CH), 131.7 (CH), |
| | | 129.2 (CH), 128.8 (CH), 128.7 (CH), 128.3 (C_{q}), 128.2 (2C, CH), 128.8 |
| | | (CH), 122.1 (CH), 108.3 (CH), 63.7 (C_{q}), 45.7 (CH₂), 26.4 (CH₃). |
| 18 | 18 | ¹H-NMR (500 MHz, CDCl₃): δ = 9.07 (br s, 1H, NH), 7.88 (d, J = 7.2 Hz, |
| | | 2H, Ar—H), 7.83 (d, J = 7.3 Hz, 2H, Ar—H), 7.51-7.48 (m, 1H, NH; 1H, |
| | | Ar—H), 7.46-7.40 (m, 3H, Ar—H), 7.30 (t, J = 7.7 Hz, 2H, Ar—H), 7.14 (d, |
| | | J = 7.8 Hz, 1H, Ar—H), 7.11 (s, 1H, Ar—H), 6.81 (d, J = 7.8 Hz, 1H, |
| | | Ar—H), 4.29 (dd, J = 14.6, 8.1 Hz, 1H, CH₂), 3.38 (dd, J = 14.5, 4.8 Hz, 1H, |
| | | CH₂), 3.27 (s, 3H, CH₃), 2.30 (s, 3H, CH₃). ¹³C{¹H}-NMR (125 MHz, |
| | | CDCl₃): δ = 175.3 (CO), 170.9 (CO), 166.3 (CO), 141.0 (C_{q}), 133.2 (C_{q}), |
| | | 132.8 (C_{q}), 132.6 (C_{q}), 132.3 (CH), 132.0 (CH), 129.9 (CH), 128.8 (2C, |
| | | CH), 128.7 (2C, CH), 128.3 (C_{q}), 127.7 (2C, CH), 127.5 (2C, CH), 123.6 |
| | | (CH), 108.5 (CH), 63.4 (C_{q}), 45.6 (CH₂), 26.8 (CH₃), 21.3 (CH₃). HRMS |
| | | (ESI): m/z Calcd for C₂₅H₂₃N₃O₃ + H⁺ [M + H]⁺ 414.1812; Found |
| | | 414.1810. |
| 19 | 19 | ¹H-NMR (500 MHz, CDCl₃): δ = 9.18 (br s, 1H, NH), 7.88 (d, J = 7.6 Hz, |
| | | 2H, Ar—H), 7.80 (d, J = 7.7 Hz, 2H, Ar—H), 7.66 (br s, 1H, NH), 7.48 (d, |
| | | J = 7.0 Hz, 1H, Ar—H), 7.44-7.40 (m, 3H, Ar—H), 7.25-7.21 (m 2H, Ar—H), |
| | | 6.90 (s, 1H, Ar—H), 6.87-6.81 (m, 2H, Ar—H), 4.26 (dd, J = 14.5, 7.8 |
| | | Hz, 1H, CH₂), 3.73 (s, 3H, OCH₃), 3.39 (dd, J = 14.5, 4.5 Hz, 1H, CH₂), |
| | | 3.25 (s, 3H, CH₃). ¹³C{¹H}-NMR (125 MHz, CDCl₃): δ = 175.1 (CO), |
| | | 170.9 (CO), 166.3 (CO), 156.5 (C_{q}), 136.7 (C_{q}), 133.0 (C_{q}), 132.5 (C_{q}), |
| | | 132.3 (CH), 132.0 (CH), 129.6 (C_{q}), 128.7 (2C, CH), 128.6 (2C, CH), |
| | | 127.7 (2C, CH), 127.5 (2C, CH), 113.9 (CH), 110.2 (CH), 109.1 (CH), |
| | | 63.7 (C_{q}), 55.9 (OCH₃), 45.7 (CH₂), 26.8 (CH₃). HRMS (ESI): m/z Calcd |
| | | for C₂₅H₂₃N₃O₄ + H⁺ [M + H]⁺ 430.1761; Found 430.1764. |
| 20 | 20 | ¹H-NMR (500 MHz, CDCl₃): δ = 9.38 (br s, 1H, NH), 7.87 (d, J = 8.1 Hz, |
| | | 2H, Ar—H), 7.84-7.77 (m, 3H, Ar—H), 7.49 (d, J = 7.1 Hz, 1H, Ar—H), |
| | | 7.44-7.37 (m, 1H, NH; 2H, Ar—H), 7.24-7.20 (m, 2H, Ar—H), 7.03-6.98 |
| | | (m, 2H, Ar—H), 6.81-6.78 (m, 1H, Ar—H), 4.15- (dd, J = 14.3, 7.5 Hz, 1H, |
| | | CH₂), 3.40 (dd, J = 14.5, 4.7 Hz, 1H, CH₂), 3.20 (s, 3H, CH₃). ¹³C{¹H}- |
| | | NMR (125 MHz, CDCl₃): δ = 175.2 (CO), 170.9 (CO), 165.5 (CO), 159.5 |
| | | (d, ¹J_{C—F} = 241.8 Hz, C_{q}), 139.2 (C_{q}), 132.8 (C_{q}), 132.4 (C_{q}), 132.3 (CH), |
| | | 132.2 (CH), 129.9 (d, ³J_{C—F} = 7.6 Hz, C_{q}), 128.7 (2C, CH), 128.6 (2C, |
| | | CH), 127.7 (2C, CH), 127.5 (2C, CH), 115.7 (d, ²J_{C—F} = 23.6 Hz, CH), |
| | | 111.1 (d, ²J_{C—F} = 25.2 Hz, CH), 109.2 (d, ³J_{C—F} = 7.6 Hz, CH), 63.6 (C_{q}), |
| | | 45.6 (CH₂), 26.8 (CH₃). ¹⁹F-NMR (376 MHz, CDCl₃): δ = −119.4 (s). |
| | | HRMS (ESI): m/z Calcd for C₂₄H₂₀FN₃O₃ + Na⁺ [M + Na]⁺ 440.1381; |
| | | Found 440.1376. |
| 21 | 21 | ¹H-NMR (400 MHz, CDCl₃): δ = 9.17 (br s, 1H, NH), 7.86-7.83 (m, 4H, |
| | | Ar—H), 7.51-7.48 (m, 1H, NH; 1H, Ar—H), 7.44-7.35 (m, 4H, Ar—H), 7.33- |
| | | 7.30 (m, 2H, Ar—H), 7.25-7.24 (m, 1H, Ar—H), 6.84 (d, J = 8.2 Hz, 1H, |
| | | Ar—H), 4.18 (dd, J = 14.6, 8.0 Hz, 1H, CH₂), 3.46 (dd, J = 14.6, 5.0 Hz, |
| | | 1H, CH₂), 3.28 (s, 3H, CH₃). ¹³C{¹H}-NMR (100 MHz, CDCl₃): δ = 174.9 |
| | | (CO), 171.0 (CO), 165.5 (CO), 141.9 (C_{q}), 132.9 (C_{q}), 132.5 (CH), 132.2 |
| | | (C_{q}), 132.1 (CH), 129.9 (C_{q}), 129.6 (CH), 128.9 (2C, CH), 128.7 (2C, |
| | | CH), 128.6 (C_{q}), 127.7 (2C, CH), 127.5 (2C, CH), 123.3 (CH), 109.7 |
| | | (CH), 63.4 (C_{q}), 45.4 (CH₂), 26.9 (CH₃). HRMS (ESI): m/z Calcd for |
| | | C₂₄H₂₀ClN₃O₃ + Na⁺ [M + Na]⁺ 456.1085; Found 456.1083. |
| 22 | 22 | ¹H-NMR (500 MHz, CDCl₃): δ = 9.05 (br s, 1H, NH), 7.85-7.80 (m, 4H, |
| | | Ar—H), 7.51-7.46 (m, 2H, Ar—H), 7.43-7.35 (m, 1H, NH; 3H, Ar—H), 7.30- |
| | | 7.28 (m, 2H, Ar—H), 7.06 (vt, J = 7.4 Hz, 1H, Ar—H), 6.92 (d, J = 7.6 Hz, |
| | | 1H, Ar—H), 4.22 (dd, J = 14.2, 8.5 Hz, 1H, CH₂), 3.48 (d, J = 14.5 Hz, |
| | | 1H, CH₂), 3.31 (s, 3H, CH₃). ¹³C{¹H}-NMR (125 MHz, CDCl₃): δ = 174.8 |
| | | (CO), 171.0 (CO), 166.5 (CO), 142.3 (C_{q}), 132.8 (C_{q}), 132.4 (2C, CH), |
| | | 132.2 (CH), 132.1 (C_{q}), 130.3 (C_{q}), 128.8 (2C, CH), 128.7 (2C, CH), |
| | | 127.7 (2C, CH), 127.5 (2C, CH), 126.0 (CH), 115.8 (C_{q}), 110.1 (CH), |
| | | 63.4 (Ca), 45.5 (CH₂), 26.8 (CH₃). HRMS (ESI): m/z Calcd for |
| | | C₂₄H₂₀BrN₃O₃ + H⁺ [M + H]⁺ 478.0761, 480.0746; Found 478.0764, |
| | | 480.0748. |
| 23 | 23 | ¹H-NMR (400 MHz, CDCl₃): δ = 9.15 (br s, 1H, NH), 7.86 (d, J = 7.2 Hz, |
| | | 2H, Ar—H), 7.80 (d, J = 7.5 Hz, 2H, Ar—H), 7.64 (br s, 1H, NH), 7.49-7.24 |
| | | (m, 8H, Ar—H), 7.02 (vt, J = 7.3 Hz, 1H, Ar—H), 6.92 (d, J = 7.7 Hz, 1H, |
| | | Ar—H), 4.21 (dd, J = 14.4, 7.7 Hz, 1H, CH₂), 3.85-3.75 (m, 2H, CH₂), 3.41 |
| | | (dd, J = 14.4, 4.4 Hz, 1H, CH₂), 1.32 (t, J = 6.9 Hz, 3H, CH₃). ¹³C{¹H}- |
| | | NMR (100 MHz, CDCl₃): δ = 174.9 (CO), 170.9 (CO), 166.3 (CO), 142.4 |
| | | (C_{q}), 133.1 (C_{q}), 132.7 (C_{q}), 132.2 (CH), 131.9 (CH), 129.5 (CH), 128.7 |
| | | (2C, CH), 128.6 (2C, CH), 127.7 (2C, CH), 127.5 (2C, CH), 127.3 (C_{q}), |
| | | 122.9 (2C, CH), 108.8 (CH), 63.3 (C_{q}), 45.7 (CH₂), 35.2 (CH₂), 12.5 |
| | | (CH₃). HRMS (ESI): m/z Calcd for C₂₅H₂₃N₃O₃ + H⁺ [M + H]⁺ 414.1812; |
| | | Found 414.1813. |
| 24 | 24 | ¹H-NMR (500 MHz, CDCl₃): δ = 9.02 (br s, 1H, NH), 7.85 (d, J = 8.0 Hz, |
| | | 4H, Ar—H), 7.49-7.45 (m, 1H, NH; 2H, Ar—H), 7.48-7.28 (m, 6H, Ar—H), |
| | | 7.02 (t, J = 7.5 Hz, 1H, Ar—H), 6.92 (d, J = 7.8 Hz, 1H, Ar—H), 4.19 (dd, |
| | | J = 14.5, 8.0 Hz, 1H, CH₂), 3.73 (t, J = 7.2 Hz, 2H, CH₂), 3.43 (dd, J = |
| | | 14.5, 4.7 Hz, 1H, CH₂), 1.84-1.74 (m, 2H, CH₂), 1.00 (t, J = 7.3 Hz, 3H, |
| | | CH₃). ¹³C{¹H}-NMR (125 MHz, CDCl₃): δ = 175.2 (CO), 170.9 (CO), |
| | | 166.3 (CO), 142.9 (C_{q}), 133.3 (C_{q}), 132.7 (C_{q}), 132.4 (CH), 131.9 (CH), |
| | | 129.5 (CH), 128.8 (2C, CH), 128.6 (2C, CH), 128.4 (C_{q}), 127.7 (2C, CH), |
| | | 127.5 (2C, CH), 122.9 (CH), 122.8 (CH), 109.0 (CH), 63.2 (C_{q}), 45.7 |
| | | (CH₂), 42.2 (CH₂), 20.9 (CH₂), 11.6 (CH₃). |
| 25 | 25 | ¹H-NMR (400 MHz, CDCl₃): δ = 9.12 (br s, 1H, NH), 7.87-7.82 (m, 4H, |
| | | Ar—H), 7.60 (br s, 1H, NH), 7.49-7.38 (m, 4H, Ar—H), 7.31 (vt, J = 7.7 |
| | | Hz, 3H, Ar—H), 7.25 (d, J = 8.2 Hz, 1H, Ar—H), 7.00 (t, J = 7.3 Hz, 1H, |
| | | Ar—H), 6.91 (d, J = 7.7 Hz, 1H, Ar—H), 4.17 (dd, J = 14.5, 7.8 Hz, 1H, |
| | | CH₂), 3.72 (t, J = 7.3 Hz, 2H, CH₂), 3.40 (dd, J = 14.5, 4.8 Hz, 1H, CH₂), |
| | | 1.76-1.67 (m, 2H, CH₂), 1.46-1.36 (m, 2H, CH₂), 0.93 (t, J = 7.3 Hz, 3H, |
| | | CH₃). ¹³C{¹H}-NMR (100 MHz, CDCl₃): δ = 175.1 (CO), 170.8 (CO), |
| | | 166.2 (CO), 142.8 (C_{q}), 133.2 (C_{q}), 132.7 (C_{q}), 132.3 (CH), 131.9 (CH), |
| | | 129.5 (CH), 128.8 (2C, CH), 128.6 (2C, CH), 128.4 (C_{q}), 127.7 (2C, CH), |
| | | 127.5 (2C, CH), 122.9 (CH), 122.8 (CH), 108.9 (CH), 63.1 (C_{q}), 45.7 |
| | | (CH₂), 40.3 (CH₂), 29.4 (CH₂), 20.4 (CH₂), 13.9 (CH₃). HRMS (ESI): |
| | | m/z Calcd for C₂₇H₂₇N₃O₃ + H⁺ [M + H]⁺ 442.2125; Found 442.2125. |
| 26 | 26 | ¹H-NMR (400 MHz, CDCl₃): δ = 9.02 (br s, 1H, NH), 7.85 (vt, J = 6.3 |
| | | Hz, 4H, Ar—H), 7.48-7.45 (m, 1H, NH; 2H, Ar—H), 7.42-7.28 (m, 6H, |
| | | Ar—H), 7.02 (vt, J = 7.4 Hz, 1H, Ar—H), 6.91 (d, J = 7.7 Hz, 1H, Ar—H), 4.19 |
| | | (dd, J = 14.5, 8.0 Hz, 1H, CH₂), 3.78-3.70 (m, 2H, CH₂), 3.42 (dd, J = |
| | | 14.5, 4.7 Hz, 1H, CH₂), 1.79-1.72 (m, 2H, CH₂), 1.42-1.24 (m, 6H, CH₂), |
| | | 0.90-0.86 (m, 3H, CH₃). ¹³C{¹H}-NMR (100 MHz, CDCl₃): δ = 175.1 |
| | | (CO), 170.9 (CO), 166.2 (CO), 142.9 (C_{q}), 133.3 (C_{q}), 132.7 (C_{q}), 132.3 |
| | | (CH), 131.9 (CH), 129.5 (CH), 128.8 (2C, CH), 128.6 (2C, CH), 128.4 |
| | | (C_{q}), 127.7 (2C, CH), 127.5 (2C, CH), 122.9 (CH), 122.8 (CH), 108.9 |
| | | (CH), 63.1 (C_{q}), 45.6 (CH₂), 40.6 (CH₂), 31.6 (CH₂), 27.3 (CH₂), 26.8 |
| | | (CH₂), 22.8 (CH₂), 14.2 (CH₃). HRMS (ESI): m/z Calcd for C₂₉H₃₁N₃O₃ + |
| | | H⁺ [M + H]⁺ 470.2438; Found 470.2440. |
| 27 | 27 | ¹H-NMR (400 MHz, CDCl₃): δ = 9.04 (br s, 1H, NH), 7.86 (d, J = 7.2 Hz, |
| | | 2H, Ar—H), 7.81 (d, J = 7.5 Hz, 2H, Ar—H), 7.49-7.38 (m, 1H, NH; 4H, |
| | | Ar—H), 7.32-7.23 (m, 6H, Ar—H), 7.20-7.15 (m, 3H, Ar—H), 7.03 (t, J = |
| | | 7.5 Hz, 1H, Ar—H), 6.80 (d, J = 7.8 Hz, 1H, Ar—H), 4.16 (dd, J = 14.5, 8.0 |
| | | Hz, 1H, CH₂), 3.83-3.69 (m, 2H, CH₂), 3.32 (dd, J = 14.5, 4.8 Hz, 1H, |
| | | CH₂), 2.82-2.68 (m, 2H, CH₂), 2.15-2.03 (m, 2H, CH₂). ¹³C{¹H}-NMR |
| | | (100 MHz, CDCl₃): δ = 175.2 (CO), 170.9 (CO), 166.2 (CO), 142.7 (C_{q}), |
| | | 141.3 (C_{q}), 133.1 (C_{q}), 132.7 (C_{q}), 132.3 (CH), 131.9 (CH), 129.5 (CH), |
| | | 128.7 (2C, CH), 128.6 (4C, CH), 128.5 (2C, CH), 128.5 (C_{q}), 127.7 (2C, |
| | | CH), 127.5 (2C, CH), 126.2 (CH), 122.9 (2C, CH), 108.8 (CH), 63.2 (C_{q}), |
| | | 45.6 (CH₂), 40.1 (CH₂), 33.3 (CH₂), 28.7 (CH₂). HRMS (ESI): m/z Calcd |
| | | for C₃₂H₂₉N₃O₃ + H⁺ [M + H]⁺ 504.2282; Found 504.2282. |
| 28 | 28 | ¹H-NMR (400 MHz, CDCl₃): δ = 8.93 (br s, 1H, NH), 7.86-7.82 (m, 4H, |
| | | Ar—H), 7.51-7.36 (m, 1H, NH, 5H, Ar—H), 7.30-7.24 (m, 3H, Ar—H), 7.07 |
| | | (t, J = 7.8 Hz, 1H, Ar—H), 6.99 (t, J = 7.3 Hz, 1H, Ar—H), 4.72-4.65 (m, |
| | | 1H, CH), 4.13 (dd, J = 14.3, 8.1 Hz, 1H, CH₂), 3.43 (dd, J = 14.6, 4.6 Hz, |
| | | 1H, CH₂), 1.56 (d, J = 6.8 Hz, 3H, CH₃), 1.52 (d, J = 7.0 Hz, 3H, CH₃). |
| | | ¹³C{¹H}-NMR (100 MHz, CDCl₃): δ = 174.9 (CO), 170.9 (CO), 162.3 |
| | | (CO), 142.2 (C_{q}), 133.7 (C_{q}), 132.8 (C_{q}), 132.4 (CH), 131.8 (CH), 129.3 |
| | | (CH), 128.9 (2C, CH), 128.7 (C_{q}), 128.6 (2C, CH), 127.7 (2C, CH), 127.5 |
| | | (2C, CH), 123.1 (CH), 122.5 (CH), 110.5 (CH), 62.8 (C_{q}), 45.5 (CH₂), |
| | | 44.4 (CH), 19.7 (CH₃), 19.3 (CH₃). HRMS (ESI): m/z Calcd for |
| | | C₂₆H₂₅N₃O₃ + H⁺ [M + H]⁺ 428.1969; Found 428.1969. |
| 29 | 29 | ¹H-NMR (400 MHz, CDCl₃): δ = 8.97 (br s, 1H, NH), 7.87 (d, J = 8.0 Hz, |
| | | 4H, Ar—H), 7.58-7.52 (m, 1H, Ar—H), 7.49-7.44 (m, 2H, Ar—H), 7.41 (br |
| | | s, 1H, NH), 7.40-7.35 (m, 3H, Ar—H), 7.10-7.08 (m, 2H, Ar—H), 6.99-6.97 |
| | | (m, 1H, Ar—H), 4.73-4.63 (m, 1H, CH), 4.26 (dd, J = 14.3, 8.1 Hz, 1H, |
| | | CH₂), 3.34 (dd, J = 14.5, 4.6 Hz, 1H, CH₂), 2.27 (s, 3H, CH₃), 1.55 (d, |
| | | J = 7.0 Hz, 3H, CH₃), 1.51 (d, J = 7.0 Hz, 3H, CH₃). ¹³C{¹H}-NMR (100 |
| | | MHz, CDCl₃): δ = 175.0 (CO), 170.9 (CO), 166.3 (CO), 139.5 (C_{q}), 133.3 |
| | | (C_{q}), 132.8 (C_{q}), 132.2 (C_{q}), 131.8 (CH), 129.6 (CH), 128.8 (2C, CH), |
| | | 128.7 (C_{q}), 128.6 (2C, CH), 127.7 (2C, CH), 127.5 (2C, CH), 130.2 (CH), |
| | | 123.9 (CH), 110.3 (CH), 62.8 (C_{q}), 45.5 (CH₂), 44.4 (CH), 21.1 (CH₃), |
| | | 19.7 (CH₃), 19.3 (CH₃). HRMS (ESI): m/z Calcd for C₂₇H₂₇N₃O₃ + H⁺ |
| | | [M + H]⁺ 442.2125; Found 442.2127. |
| 30 | 30 | ¹H-NMR (400 MHz, CDCl₃): δ = 8.97 (br s, 1H, NH), 7.87-7.81 (m, 4H, |
| | | Ar—H), 7.55-7.51 (m, 1H, Ar—H), 7.50-7.38 (m, 5H, Ar—H), 7.21 (br s, |
| | | 1H, NH), 7.03-6.94 (m, 3H, Ar—H), 4.68 (septet, J = 7.0 Hz, 1H, CH), |
| | | 4.10 (dd, J = 14.5, 8.0 Hz, 1H, CH₂), 3.49 (dd, J = 14.5, 4.9 Hz, 1H, CH₂), |
| | | 1.56 (d, J = 7.0 Hz, 3H, CH₃), 1.53 (d, J = 7.0 Hz, 3H, CH₃). ¹³C{¹H}- |
| | | NMR (100 MHz, CDCl₃): δ = 174.8 (CO), 171.0 (CO), 166.5 (CO), 159.1 |
| | | (d, ¹J_{C—F} = 241.8 Hz, C_{q}), 138.0 (d, ⁴J_{C—F} = 2.29 Hz, C_{q}), 132.8 (d, ³J_{C—F} = |
| | | 70.2 Hz, C_{q}), 132.5 (CH), 132.0 (CH), 130.6 (C_{q}), 130.5 (C_{q}), 128.9 (2C, |
| | | CH), 128.7 (2C, CH), 127.7 (2C, CH), 127.5 (2C, CH), 115.5 (d, ²J_{C—F} = |
| | | 23.6 Hz, CH), 111.2 (d, ²J_{C—F} = 32.8 Hz, CH), 111.1 (CH), 63.0 (C_{q}), 45.4 |
| | | (CH₂), 44.6 (CH), 19.7 (CH₃), 19.3 (CH₃). ¹⁹F-NMR (376 MHz, CDCl₃): |
| | | δ = −120.6 (s). HRMS (ESI): m/z Calcd for C₂₆H₂₄FN₃O₃ + H⁺ [M + H]⁺ |
| | | 446.1874; Found 446.1873. |
| 31 | 31 | ¹H-NMR (400 MHz, CDCl₃): δ = 9.03 (br s, 1H, NH), 7.87-7.83 (m, 4H, |
| | | Ar—H), 7.53-7.45 (m, 1H, NH; 1H, Ar—H), 7.42-7.36 (m, 6H, Ar—H), 7.28- |
| | | 7.27 (m, 1H, Ar—H), 6.95 (d, J = 8.4 Hz, 1H, Ar—H), 4.65 (septet, J = 7.0 |
| | | Hz, 1H, CH), 4.11 (dd, J = 14.5, 8.0 Hz, 1H, CH₂), 3.46-3.43 (m, 1H, |
| | | CH₂), 1.54 (d, J = 6.9 Hz, 3H, CH₃), 1.50 (d, J = 7.0 Hz, 3H, CH₃). |
| | | ¹³C{¹H}-NMR (100 MHz, CDCl₃): δ = 174.5 (CO), 171.0 (CO), 166.4 |
| | | (CO), 141.2 (C_{q}), 133.2 (C_{q}), 132.5 (CH), 132.4 (C_{q},), 132.1 (CH), 132.0 |
| | | (CH), 130.9 (C_{q}), 128.9 (2C, CH), 128.7 (2C, CH), 127.7 (2C, CH), 127.5 |
| | | (2C, CH), 126.3 (CH), 115.3 (C_{q}), 111.9 (CH), 62.8 (C_{q}), 45.4 (CH₂), 44.6 |
| | | (CH), 19.6 (CH₃), 19.2 (CH₃). HRMS (ESI): m/z Calcd for C₂₆H₂₄BrN₃O₃ + |
| | | H⁺ [M + H]⁺ 506.1074; Found 506.1077. |
| 32 | 32 | ¹H-NMR (500 MHz, CDCl₃): δ = 8.92 (br s, 1H, NH), 7.88 (d, J = 7.2 Hz, |
| | | 2H, Ar—H), 7.83 (d, J = 7.2 Hz, 2H, Ar—H), 7.57-7.54 (m, 1H, Ar—H), |
| | | 7.49-7.45 (m, 1H, NH; 2H, Ar—H), 7.42-7.38 (m, 2H, Ar—H), 7.29-7.28 |
| | | (m, 2H, Ar—H), 7.07-7.01 (m, 2H, Ar—H), 4.69 (septet, J = 7.0 Hz, 1H, |
| | | CH), 4.11 (dd, J = 14.6, 8.2 Hz, 1H, CH₂), 3.50 (dd, J = 14.6, 4.9 Hz, 1H, |
| | | CH₂), 1.57 (d, J = 7.0 Hz, 3H, CH₃), 1.54 (d, J = 7.0 Hz, 3H, CH₃). |
| | | ¹³C{¹H}-NMR (125 MHz, CDCl₃): δ = 174.6 (CO), 171.0 (CO), 166.5 |
| | | (CO), 140.6 (C_{q}), 133.1 (C_{q}), 132.5 (C_{q}), 132.4 (CH), 132.0 (CH), 130.5 |
| | | (C_{q}), 129.2 (CH), 128.9 (2C, CH), 128.6 (2C, CH), 128.0 (C_{q}), 127.7 (2C, |
| | | CH), 127.5 (2C, CH), 123.7 (CH), 111.4 (CH), 62.9 (Cq), 45.4 (CH₂), 44.6 |
| | | (CH), 19.7 (CH₃), 19.2 (CH₃). HRMS (ESI): m/z Calcd for C₂₆H₂₄ClN₃O₃ + |
| | | H⁺ [M + H]⁺ 462.1579; Found 462.1579. |
| 33 | 33 | ¹H-NMR (400 MHz, CDCl₃): δ = 8.92 (br s, 1H, NH), 7.76 (d, J = 7.6 Hz, |
| | | 4H, Ar—H), 7.41-7.35 (m, 1H, NH; 2H, Ar—H), 7.32-7.28 (m, 4H, Ar—H), |
| | | 7.21 (t, J = 7.7 Hz, 1H, Ar—H), 7.15 (d, J = 7.3 Hz, 1H, Ar—H), 6.91 (t, |
| | | J = 7.5 Hz, 1H, Ar—H), 6.82 (d, J = 7.8 Hz, 1H, Ar—H), 4.06 (dd, J = 14.5, |
| | | 8.0 Hz, 1H, CH₂), 3.54-3.48 (m, 1H, CH₂), 3.45-3.40 (m, 1H, CH₂), 3.32 |
| | | (dd, J = 14.5, 4.6 Hz, 1H, CH₂), 2.15-2.08 (m, 1H, CH), 0.94 (d, J = 6.6 |
| | | Hz, 3H, CH₃), 0.88 (d, J = 6.6 Hz, 3H, CH₃). ¹³C{¹H}-NMR (100 MHz, |
| | | CDCl₃): δ = 175.3 (CO), 170.7 (CO), 166.2 (CO), 143.2 (C_{q}), 133.3 (C_{q}), |
| | | 132.8 (C_{q}), 132.3 (CH), 131.9 (CH), 129.4 (CH), 128.8 (2C, CH), 128.6 |
| | | (2C, CH), 128.3 (C_{q}), 127.7 (2C, CH), 127.5 (2C, CH), 122.8 (2C, CH), |
| | | 109.1 (CH), 63.0 (C_{q}), 48.1 (CH₂), 45.8 (CH₂), 27.4 (CH), 20.5 (2C, CH₃). |
| | | HRMS (ESI): m/z Calcd for C₂₇H₂₇N₃O₃ + H⁺ [M + H]⁺ 442.2125; Found |
| | | 442.2127. |
| 34 | 34 | ¹H-NMR (400 MHz, CDCl₃): δ = 9.21 (br s, 1H, NH), 7.86 (d, J = 7.1 Hz, |
| | | 2H, Ar—H), 7.81 (d, J = 7.2 Hz, 2H, Ar—H), 7.60 (br s, 1H, NH), 7.49-7.45 |
| | | (m, 1H, Ar—H), 7.43-7.38 (m, 3H, Ar—H), 7.30-7.25 (m, 4H, Ar—H), 7.01 |
| | | (t, J = 7.5 Hz, 1H, Ar—H), 6.88 (d, J = 7.7 Hz, 1H, Ar—H), 5.92-5.82 (m, |
| | | 1H, CH), 5.39 (d, J = 17.2 Hz, 1H, CH₂), 5.22 (d, J = 10.4 Hz, 1H, CH₂), |
| | | 4.37 (d, J = 5.0 Hz, 2H, CH₂), 4.19 (dd, J = 14.5, 7.7 Hz, 1H, CH₂), 3.42 |
| | | (dd, J = 14.5, 5.0 Hz, 1H, CH₂). ¹³C{¹H}-NMR (100 MHz, CDCl₃): δ = |
| | | 175.1 (CO), 170.9 (CO), 166.3 (CO), 142.4 (C_{q}), 132.9 (C_{q}), 132.6 (C_{q}), |
| | | 132.2 (CH), 131.9 (CH), 131.2 (CH), 129.4 (CH), 128.6 (2C, CH), 128.5 |
| | | (2C, CH), 128.3 (C_{q}), 127.7 (2C, CH), 127.5 (2C, CH), 123.1 (CH), 122.7 |
| | | (CH), 117.9 (CH₂), 109.6 (CH), 63.3 (C_{q}), 45.7 (CH₂), 42.8 (CH₂). HRMS |
| | | (ESI): m/z Calcd for C₂₆H₂₃N₃O₃⁺ [M]⁺ 425.1734; Found 425.1721. |
| 35 | 35 | ¹H-NMR (500 MHz, CDCl₃): δ = 9.02 (br s, 1H, NH), 7.75-7.69 (m, 4H, |
| | | Ar—H), 7.52 (br s, 1H, NH), 7.36-7.28 (m, 4H, Ar—H), 7.20-7.11 (m, 4H, |
| | | Ar—H), 7.86 (d, J = 8.1 Hz, 2H, Ar—H), 4.63 (septet, J = 8.4 Hz, 1H, CH), |
| | | 4.05-3.98 (m, 1H, CH₂), 3.25-3.21 (m, 1H, CH₂), 2.09-1.98 (m, 2H, CH₂), |
| | | 1.79 (s, 4H, CH₂), 1.54 (s, 2H, CH₂). ¹³C{¹H}-NMR (125 MHz, CDCl₃): |
| | | δ = 175.3 (CO), 170.9 (CO), 166.3 (CO), 142.1 (C_{q}), 133.2 (C_{q}), 132.7 |
| | | (C_{q}), 132.2 (CH), 131.8 (CH), 129.2 (CH), 128.7 (2C, CH), 128.6 (1C, |
| | | C_{q}; 2C, CH), 127.7 (2C, CH), 127.5 (2C, CH), 122.9 (CH), 122.6 (CH), |
| | | 110.1 (CH), 62.9 (C_{q}), 53.0 (CH), 45.6 (CH₂), 28.1 (CH₂), 27.6 (CH₂), |
| | | 25.4 (2C, CH₂). HRMS (ESI): m/z Calcd for C₂₈H₂₇N₃O₃ + H⁺ [M + H]⁺ |
| | | 454.2125; Found 454.2147. |
| 36 | 36 | ¹H-NMR (400 MHz, CDCl₃): δ = 9.04 (br s, 1H, NH), 7.86 (d, J = 7.2 Hz, |
| | | 2H, Ar—H), 7.81 (d, J = 7.5 Hz, 2H, Ar—H), 7.49-7.38 (m, 1H, NH; 4H, |
| | | Ar—H), 7.32-7.23 (m, 6H, Ar—H), 7.20-7.15 (m, 3H, Ar—H), 7.03 (t, J = |
| | | 7.5 Hz, 1H, Ar—H), 6.80 (d, J = 7.8 Hz, 1H, Ar—H), 4.16 (dd, J = 14.5, 8.0 |
| | | Hz, 1H, CH₂), 3.83-3.69 (m, 2H, CH₂), 3.32 (dd, J = 14.5, 4.8 Hz, 1H, |
| | | CH₂), 2.82-2.68 (m, 2H, CH₂), 2.15-2.03 (m, 2H, CH₂). ¹³C{¹H}-NMR |
| | | (100 MHz, CDCl₃): δ = 175.2 (CO), 170.9 (CO), 166.2 (CO), 142.7 (C_{q}), |
| | | 141.3 (C_{q}), 133.1 (C_{q}), 132.7 (C_{q}), 132.3 (CH), 131.9 (CH), 129.5 (CH), |
| | | 128.7 (2C, CH), 128.6 (4C, CH), 128.5 (2C, CH), 128.5 (C_{q}), 127.7 (2C, |
| | | CH), 127.5 (2C, CH), 126.2 (CH), 122.9 (2C, CH), 108.8 (CH), 63.2 (C_{q}), |
| | | 45.6 (CH₂), 40.1 (CH₂), 33.3 (CH₂), 28.7 (CH₂). HRMS (ESI): m/z Calcd |
| | | for C₃₂H₂₉N₃O₃ + H⁺ [M + H]⁺ 504.2282; Found 504.2282. |
| 37 | 37 | ¹H-NMR (500 MHz, CDCl₃): δ = 9.11 (br s, 1H, NH), 7.84 (t, J = 7.2 Hz, |
| | | 4H, Ar—H), 7.48-7.39 (m, 1H, NH; 4H, Ar—H), 7.38-7.28 (m, 3H, Ar—H), |
| | | 7.24 (d, J = 7.5 Hz, 1H, Ar—H), 7.02 (d, J = 7.2 Hz, 2H, Ar—H), 4.16-4.03 |
| | | (m, 2H, CH₂), 3.88-3.82 (m, 1H, CH₂), 3.72-3.61 (m, 2H, CH₂), 3.49 (dd, |
| | | J = 14.5, 4.8 Hz, 1H, CH₂), 3.34 (s, 3H, OCH₃). ¹³C{¹H}-NMR (125 MHz, |
| | | CDCl₃): δ = 175.4 (CO), 170.9 (CO), 166.3 (CO), 142.9 (C_{q}), 133.2 (C_{q}), |
| | | 132.6 (C_{q}), 132.3 (CH), 131.9 (CH), 129.5 (CH), 128.8 (2C, CH), 128.6 |
| | | (2C, CH), 128.2 (C_{q}), 127.7 (2C, CH), 127.5 (2C, CH), 122.9 (CH), 122.8 |
| | | (CH), 109.4 (CH), 69.6 (CH₂), 63.2 (C_{q}), 59.1 (OCH₃), 45.6 (CH₂), 40.3 |
| | | (CH₂). HRMS (ESI): m/z Calcd for C₂₆H₂₅N₃O₄ + H⁺ [M + H]⁺ 444.1918; |
| | | Found 444.1916. |
| 38 | 38 | ¹H-NMR (400 MHz, CDCl₃): δ = 9.16 (br s, 1H, NH), 7.87 (d, J = 7.7 Hz, |
| | | 2H, Ar—H), 7.82 (d, J = 7.8 Hz, 2H, Ar—H), 7.65 (br s, 1H, NH), 7.49-7.39 |
| | | (m, 4H, Ar—H), 7.33-7.25 (m, 4H, Ar—H), 7.01 (t, J = 7.5 Hz, 1H, Ar—H), |
| | | 6.90 (d, J = 7.7 Hz, 1H, Ar—H), 4.19 (dd, J = 14.5, 7.7 Hz, 1H, CH₂), 3.70 |
| | | (t, J = 7.3 Hz, 2H, CH₂), 3.38 (dd, J = 14.3, 4.5 Hz, 1H, CH₂), 1.79-1.68 |
| | | (m, 2H, CH₂), 1.40-1.26 (m, 10H, CH₂), 0.87 (t, J = 6.5 Hz, 3H, CH₃). |
| | | ¹³C{¹H}-NMR (100 MHz, CDCl₃): δ = 175.1 (CO), 170.9 (CO), 166.2 |
| | | (CO), 142.7 (C_{q}), 133.1 (C_{q}), 132.6 (C_{q}), 132.3 (CH), 131.9 (CH), 129.5 |
| | | (CH), 128.7 (2C, CH), 128.6 (2C, CH), 128.4 (C_{q}), 127.7 (2C, CH), 127.5 |
| | | (2C, CH), 122.9 (2C, CH), 108.9 (CH), 63.2 (C_{q}), 45.7 (CH₂), 40.6 (CH₂), |
| | | 31.9 (CH₂), 29.4 (CH₂), 29.3 (CH₂), 27.3 (CH₂), 27.1 (CH₂), 22.8 (CH₂), |
| | | 14.3 (CH₃). HRMS (ESI): m/z Calcd for C₃₁H₃₅N₃O₃ + H⁺ [M + H]⁺ |
| | | 498.2751; Found 498.2744. |
| 39 | 39 | ¹H-NMR (500 MHz, CDCl₃): δ = 9.17 (br s, 1H, NH), 8.06 (br s, 1H, NH), |
| | | 7.94 (d, J = 7.7 Hz, 1H, Ar—H), 7.89-7.88 (m, 4H, Ar—H), 7.71 (d, J = 7.6 |
| | | Hz, 1H, Ar—H), 7.53-7.50 (m, 1H, Ar—H), 7.48-7.39 (m, 6H, Ar—H), 7.27 |
| | | (vt, J = 6.8 Hz, 2H, Ar—H), 7.19 (t, J = 7.7 Hz, 1H, Ar—H), 7.00 (t, J = 6.9 |
| | | Hz, 1H, Ar—H), 6.69 (d, J = 7.5 Hz, 1H, Ar—H), 5.05 (s, 2H, CH₂), 4.16 |
| | | (dd, J = 14.5, 7.9 Hz, 1H, CH₂), 3.91 (s, 3H, OCH₃), 3.64-3.60 (m, 1H, |
| | | CH₂). ¹³C{¹H}-NMR (125 MHz, CDCl₃): δ = 175.2 (CO), 171.1 (CO), |
| | | 167.1 (CO), 166.3 (CO), 142.2 (C_{q}), 136.4 (C_{q}), 133.2 (C_{q}), 132.6 (C_{q}), |
| | | 132.5 (CH), 132.0 (CH), 131.9 (CH), 130.7 (C_{q}), 129.5 (CH), 129.4 (CH), |
| | | 128.2 (CH), 128.9 (2C, CH), 128.6 (2C, CH), 128.4 (CH), 128.2 (C_{q}), |
| | | 127.7 (2C, CH), 127.5 (2C, CH), 123.3 (CH), 122.9 (CH), 109.7 (CH), |
| | | 63.5 (C_{q}), 52.4 (OCH₃), 45.6 (CH₂), 43.9 (CH₂). HRMS (ESI): m/z Calcd |
| | | for C₃₂H₂₇N₃O₅ + H⁺ [M + H]⁺ 534.2023; Found 534.2020. |
| 40 | 40 | ¹H-NMR (400 MHz, CDCl₃): δ = 9.06 (br s, 1H, NH), 7.86-7.81 (m, 4H, |
| | | Ar—H), 7.60 (d, J = 7.9 Hz, 1H, Ar—H), 7.51-7.45 (m, 2H, Ar—H), 7.41- |
| | | 7.33 (m, 1H, NH; 4H, Ar—H), 7.29-7.23 (m, 3H, Ar—H), 7.17-7.05 (m, |
| | | 3H, Ar—H), 7.00 (t, J = 7.4 Hz, 1H, Ar—H), 6.77 (d, J = 7.7 Hz, 1H, |
| | | Ar—H), 6.48-6.45 (m, 1H, Ar—H), 4.22-4.11 (m, 2H, CH₂), 4.06 (dd, J = 14.4, |
| | | 7.9 Hz, 1H, CH₂), 3.84-3.77 (m, 1H, CH₂), 3.70-3.62 (m, 1H, CH₂), 3.38 |
| | | (dd, J = 14.4, 4.6 Hz, 1H, CH₂), 2.06-1.92 (m, 2H, CH₂), 1.84-1.67 (m, |
| | | 2H, CH₂). ¹³C{¹H}-NMR (100 MHz, CDCl₃): δ = 175.2 (CO), 170.9 |
| | | (CO), 166.2 (CO), 142.5 (C_{q}), 136.1 (C_{q}), 133.2 (C_{q}), 132.6 (C_{q}), 132.4 |
| | | (CH), 131.9 (CH), 129.6 (CH), 128.9 (2C, CH), 128.8 (C_{q}), 128.7 (CH), |
| | | 128.6 (2C, CH), 128.3 (C_{q}), 128.0 (CH), 127.7 (2C, CH), 127.5 (2C, CH), |
| | | 122.9 (CH), 121.5 (CH), 121.1 (CH), 119.3 (CH), 109.6 (CH), 108.7 |
| | | (CH), 101.2 (CH), 63.2 (C_{q}), 45.9 (CH₂), 45.6 (CH₂), 39.8 (CH₂), 27.8 |
| | | (CH₂), 24.8 (CH₂). HRMS (ESI): m/z Calcd for C₃₅H₃₂N₄O₃ + H⁺ [M + |
| | | H]⁺ 557.2547; Found 557.2548. |
| 41 | 41 | ¹H-NMR (400 MHz, CDCl₃): δ = 9.09 (br s, 1H, NH), 7.79-7.74 (m, 4H, |
| | | Ar—H), 7.54 (br s, 1H, NH), 7.41-7.36 (m, 2H, Ar—H), 7.33 (d, J = 7.6 Hz, |
| | | 2H, Ar—H), 7.24-7.20 (m, 3H, Ar—H), 7.17 (d, J = 8.2 Hz, 1H, Ar—H), |
| | | 6.93 (t, J = 7.5 Hz, 1H, Ar—H), 6.86 (d, J = 7.8 Hz, 1H, Ar—H), 5.26 (s, |
| | | 1H, CH), 4.09 (dd, J = 14.5, 7.8 Hz, 1H, CH₂), 3.70-3.66 (m, 2H, CH₂), |
| | | 3.44-3.36 (m, 2H, CH₂), 3.32 (dd, J = 14.5, 4.8 Hz, 1H, CH₂), 3.08-3.01 |
| | | (m, 1H, CH₂), 2.28-2.25 (m, 1H, CH₂), 2.13-2.06 (m, 1H, CH), 1.96-1.71 |
| | | (m, 7H), 1.60-1.17 (m, 17H), 1.11-0.97 (m, 6H), 0.91 (s, 3H, CH₃), 0.84 |
| | | (d, J = 6.5 Hz, 3H, CH₃), 0.79 (d, J = 1.7 Hz, 3H, CH₃), 0.78 (d, J = 1.7 |
| | | Hz, 3H, CH₃) 0.60 (s, 3H, CH₃). ¹³C{¹H}-NMR (100 MHz, CDCl₃): δ = |
| | | 175.1 (CO), 170.8 (CO), 166.2 (CO), 142.7 (C_{q}), 141.0 (C_{q}), 133.1 (C_{q}), |
| | | 132.6 (C_{q}), 132.3 (CH), 131.9 (CH), 129.5 (CH), 128.7 (2C, CH), 128.6 |
| | | (2C, CH), 128.4 (C_{q}), 127.7 (2C, CH), 127.5 (2C, CH), 122.9 (CH), 121.7 |
| | | (CH), 109.0 (CH), 79.1 (CH), 67.4 (CH₂), 63.2 (C_{q}), 56.9 (CH), 56.3 |
| | | (CH), 50.3 (CH), 45.7 (CH₂), 42.4 (Ca), 40.3 (CH₂), 39.9 (CH₂), 39.6 |
| | | (CH₂), 39.3 (CH₂), 37.4 (C_{q}), 37.0 (CH₂), 36.3 (CH₂), 35.9 (CH), 32.1 |
| | | (CH₂), 32.0 (CH₃), 28.6 (CH₂), 28.4 (CH₂), 28.1 (CH), 27.5 (CH₂), 24.4 |
| | | (CH₂), 24.2 (CH₂), 23.9 (CH₂), 22.9 (CH₃), 22.7 (2C, CH), 21.2 (CH₂), |
| | | 19.5 (CH₃), 18.9 (CH₃), 11.9 (CH₃). HRMS (ESI): m/z Calcd for |
| | | C₅₄H₇₁N₃O₄ + K⁺ [M + K]⁺ 864.5181; Found 864.5181. |
| 42 | 42 | ¹H-NMR (500 MHz, CDCl₃): δ = 9.04 (br s, 1H, NH), 7.85 (t, J = 7.6 Hz, |
| | | 4H, Ar—H), 7.52 (d, J = 7.0 Hz, 2H, Ar—H), 7.50 (q, J = 7.6 Hz, 2H, |
| | | Ar—H), 7.42-7.32 (m, 1H, NH; 7H, Ar—H), 7.30-7.27 (m, 4H, Ar—H), 7.03 (q, |
| | | J = 7.0 Hz, 2H, Ar—H), 6.96 (d, J = 8.2 Hz, 1H, Ar—H), 6.80 (d, J = 7.9 |
| | | Hz, 1H, Ar—H), 4.09 (dd, J = 14.6, 7.9 Hz, 1H, CH₂), 4.06-3.97 (m, 2H, |
| | | CH₂), 3.79-3.72 (m, 2H, CH₂), 3.40 (dd, J = 14.6, 4.8 Hz, 1H, CH₂), 1.91- |
| | | 1.82 (m, 4H, CH₂). ¹³C{¹H}-NMR (125 MHz, CDCl₃): δ = 175.1 (CO), |
| | | 170.9 (CO), 166.2 (CO), 155.9 (C_{q}), 142.7 (C_{q}), 138.8 (C_{q}), 133.3 (C_{q}), |
| | | 132.7 (C_{q}), 132.4 (CH), 131.9 (CH), 131.0 (CH), 129.7 (2C, CH), 129.6 |
| | | (CH), 128.9 (2C, CH), 128.8 (CH), 128.6 (2C, CH), 128.4 (2C, C_{q}), 127.9 |
| | | (3C, CH), 127.7 (2C, CH), 127.4 (2C, CH), 126.7 (CH), 122.9 (CH), |
| | | 121.1 (CH), 112.7 (CH), 108.9 (CH), 67.8 (CH₂), 63.2 (C_{q}), 45.6 (CH₂), |
| | | 40.1 (CH₂), 26.7 (CH₂), 24.2 (CH₂). |
| 43 | 43 | ¹H-NMR (400 MHz, CDCl₃): δ = 9.02 (br s, 1H, NH), 7.85-7.83 (m, 4H, |
| | | Ar—H), 7.51-7.44 (m, 1H, NH; 1H, Ar—H), 7.41-7.35 (m, 4H, Ar—H), 7.32- |
| | | 7.25 (m, 3H, Ar—H), 7.02 (t, J = 7.5 Hz, 1H, Ar—H), 6.96 (d, J = 7.7 Hz, |
| | | 1H, Ar—H), 6.80 (d, J = 8.1 Hz, 1H, Ar—H), 6.70-6.68 (m, 2H, Ar—H), |
| | | 6.00-5.90 (m, 1H, CH), 5.09-5.04 (m, 2H, CH₂), 4.16 (dd, J = 14.5, 8.0 |
| | | Hz, 1H, CH₂), 4.09-4.00 (m, 2H, CH₂), 3.89-3.84 (m, 2H, CH₂), 3.81 (s, |
| | | 3H, OCH₃), 3.44 (dd, J = 14.5, 4.9 Hz, 1H, CH₂), 3.31 (d, J = 6.6 Hz, 2H, |
| | | CH₂), 1.96-1.95 (m, 4H, CH₂). ¹³C{¹H}-NMR (100 MHz, CDCl₃): δ = |
| | | 175.2 (CO), 170.9 (CO), 166.2 (CO), 149.5 (C_{q}), 146.8 (C_{q}), 142.7 (C_{q}), |
| | | 137.8 (CH), 133.2 (C_{q}), 133.1 (C_{q}), 132.7 (C_{q}), 132.3 (CH), 131.9 (CH), |
| | | 129.5 (CH), 128.8 (2C, CH), 128.6 (2C, CH), 128.4 (C_{q}), 127.7 (2C, CH), |
| | | 127.5 (2C, CH), 127.3 (CH), 122.9 (CH), 120.7 (CH), 115.8 (CH₂), 113.7 |
| | | (CH), 112.6 (CH), 109.0 (CH), 68.7 (CH₂), 63.2 (C_{q}), 56.0 (OCH₃), 45.7 |
| | | (CH₂), 40.1 (CH₂), 39.9 (CH₂), 26.7 (CH₂), 24.1 (CH₂). HRMS (ESI): m/z |
| | | Calcd for C₃₇H₃₇N₃O₅ + H⁺ [M + H]⁺ 604.2806; Found 604.2833. |
| 44 | 44 | ¹H-NMR (400 MHz, CDCl₃): δ = 7.81 (d, J = 7.1 Hz, 2H, Ar—H), 7.52- |
| | | 7.48 (m, 1H, Ar—H), 7.45-7.39 (m, 3H, Ar—H), 7.35-7.31 (m, 1H, Ar—H), |
| | | 7.28 (br s, 1H, NH), 7.08 (vt, J = 7.5 Hz, 1H, Ar—H), 6.87 (d, J = 7.7 Hz, |
| | | 1H, Ar—H), 4.15 (dd, J = 13.7, 8.6 Hz, 1H, CH₂), 3.30 (dd, J = 13.6, 2.7 |
| | | Hz, 1H, CH₂), 3.21 (s, 3H, CH₃), 2.08 (br s, 2H, NH2). ¹³C{¹H}-NMR |
| | | (100 MHz, CDCl₃): δ = 179.3 (CO), 167.8 (CO), 143.0 (C_{q}), 134.3 (C_{q}), |
| | | 131.7 (CH), 130.1 (C_{q}), 129.8 (CH), 128.7 (2C, CH), 127.2 (2C, CH), |
| | | 123.9 (CH), 123.5 (CH), 108.8 (CH), 59.9 (C_{q}), 46.2 (CH₂), 26.1 (CH₃). |
| | | HRMS (ESI): m/z Calcd for C₁₇H₁₇N₃O₂ + H⁺ [M + H]⁺ 296.1394; Found |
| | | 296.1392. |
| 45 | 45 | ¹H-NMR (400 MHz, CDCl₃): δ = 9.21 (br s, 1H, NH), 7.87 (d, J = 7.2 Hz, |
| | | 2H, Ar—H), 7.80 (d, J = 7.3 Hz, 2H, Ar—H), 7.58 (br s, 1H, NH), 7.47 (d, |
| | | J = 7.2 Hz, 1H, Ar—H), 7.43-7.40 (m, 3H, Ar—H), 7.34 (t, J = 7.7 Hz, 1H, |
| | | Ar—H), 7.27-7.23 (m, 3H, Ar—H), 7.05 (t, J = 7.3 Hz, 1H, Ar—H), 6.90 (d, |
| | | J = 7.6 Hz, 1H, Ar—H), 3.27 (s, 3H, CH₃). ¹³C{¹H}-NMR (100 MHz, |
| | | CDCl₃): δ = 175.4 (CO), 170.9 (CO), 166.3 (CO), 143.4 (C_{q}), 133.1 (C_{q}), |
| | | 132.5 (C_{q}), 132.3 (CH), 132.0 (CH), 129.7 (CH), 128.8 (2C, CH), 128.7 |
| | | (2C, CH), 128.3 (C_{q}), 127.7 (2C, CH), 127.5 (2C, CH), 123.2 (CH), 122.8 |
| | | (CH), 108.7 (CH), 63.3 (C_{q}), 26.7 (CH₃). ²H-NMR (400 MHz, CHCl₃): δ = |
| | | 4.19 (s, 1D, CD), 3.45 (s, 1D, CD). HRMS (ESI): m/z Calcd for |
| | | C₂₄H₁₉D₂N₃O₃ + H⁺ [M + H]⁺ 402.1781; Found 402.1775. |

### Advantages of the Invention

Provides an efficient and straightforward method for generating quaternary stereogenic center by replacing sensitive nucleophiles.
Easy and simple way provided to obtain desired quaternary stereogenic center containing compounds such as stereogenic 3,3-disubstituted 3-aminooxindole compounds of formula (I), with better yields up to 90%.
Efficiently provides bio-relevant C3-aminated oxindole compounds.
It generates a quaternary carbon center with —NHR (amino) and —CH₂—NHR (aminomethyl) units, where the amino-functionality is generated through the tandem reaction with N-methoxy benzamide. These acyclic amino and aminomethyl units might generate different chemical features and can serve as a unique precursor for further development.

1. A stereogenic 3,3-disubstituted 3-aminooxindole compound of formula (I): 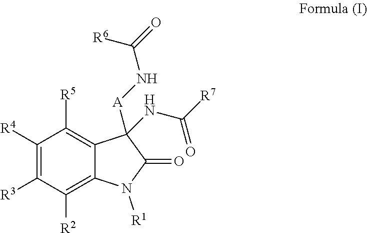
or an isomer, solvate, or tautomer thereof,
wherein
A is —CHR—, or —CD₂;
R is hydrogen, or unsubstituted or substituted C₁₋₆alkyl;
R¹ is unsubstituted or substituted C₁₋₈alkyl, unsubstituted or substituted aryl, unsubstituted or substituted alkylaryl, unsubstituted or substituted cycloalkyl, unsubstituted or substituted alkylalkoxy, unsubstituted or substituted alkoxyaryl, unsubstituted or substituted heteroaryl, unsubstituted or substituted alkylheteroaryl, unsubstituted or substituted alkoxyheteroaryl, unsubstituted or substituted ester, unsubstituted or substituted alkyloxy-cycloalkenealkyl, unsubstituted or substituted alkyloxy-aryl, unsubstituted or substituted alkyloxy-heteroaryl, unsubstituted or substituted alkyloxy-arylalkene, or unsubstituted or substituted alkene,
R² is hydrogen, halogen, unsubstituted or substituted C₁₋₆ alkyl, unsubstituted or substituted aryl, or unsubstituted or substituted heteroaryl,
R³ is hydrogen, halogen, unsubstituted or substituted C₁₋₆ alkyl, unsubstituted or substituted aryl, or unsubstituted or substituted heteroaryl,
R⁴ is hydrogen, halogen, unsubstituted or substituted C₁₋₆ alkyl, unsubstituted or substituted alkoxy, unsubstituted or substituted aryl, or unsubstituted or substituted heteroaryl,
R⁵ is hydrogen, halogen, unsubstituted or substituted C₁₋₆ alkyl, unsubstituted or substituted aryl, or unsubstituted or substituted heteroaryl,
R⁶ is unsubstituted or substituted aryl, or unsubstituted or substituted heteroaryl, and
R⁷ is unsubstituted or substituted aryl, or unsubstituted or substituted heteroaryl.

2. The compound as claimed in claim 1, wherein the compound is selected from the group consisting of:

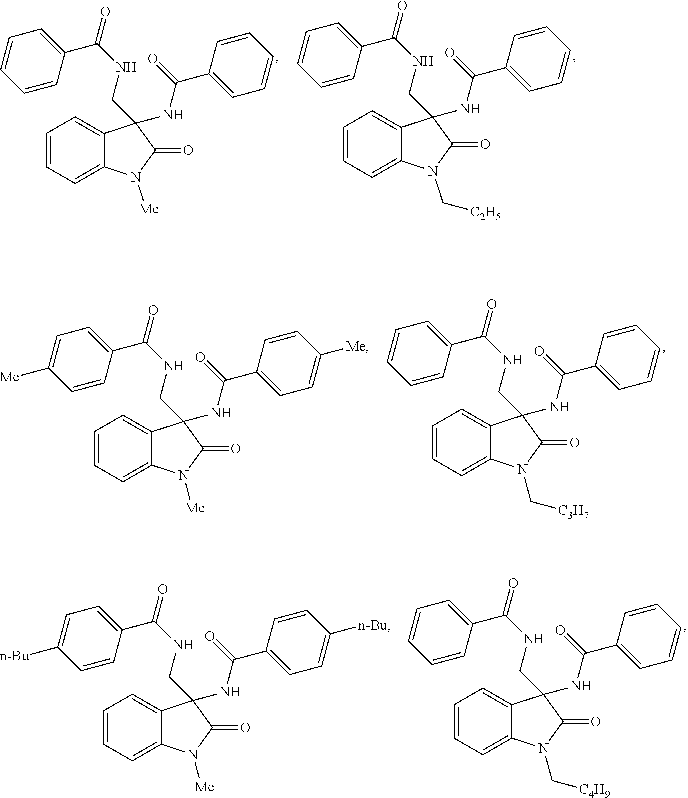

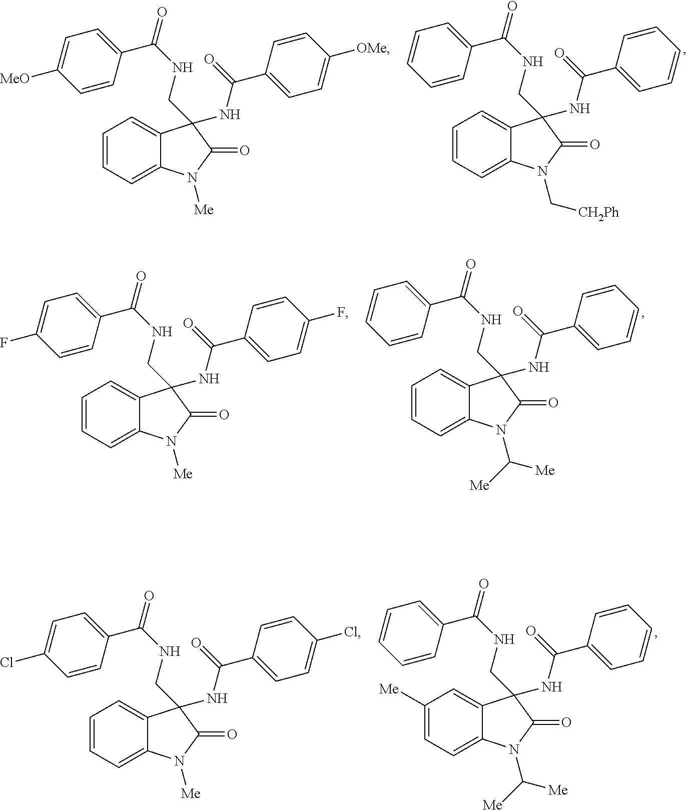

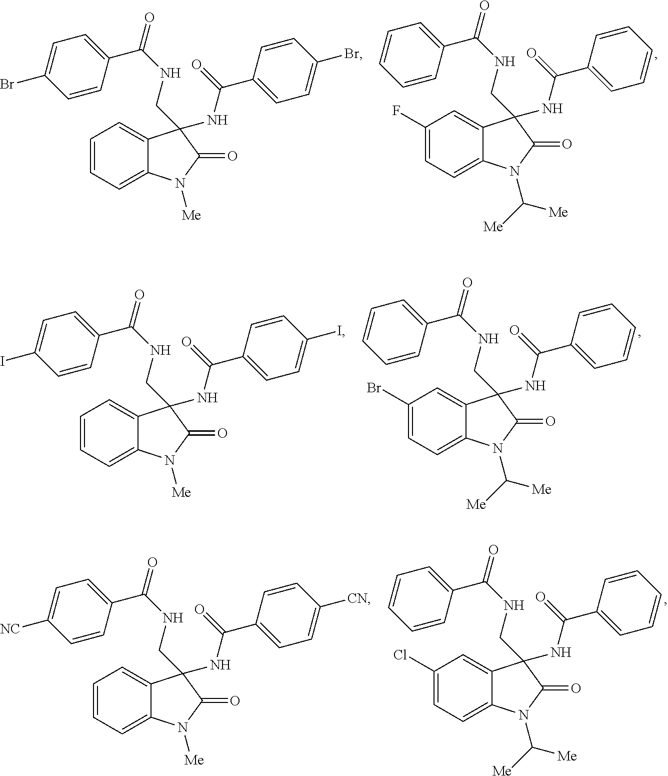

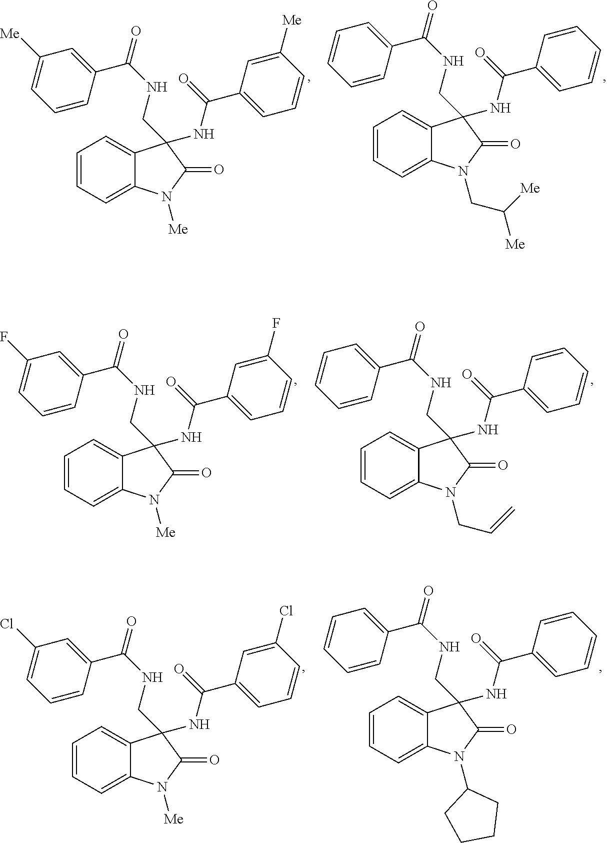

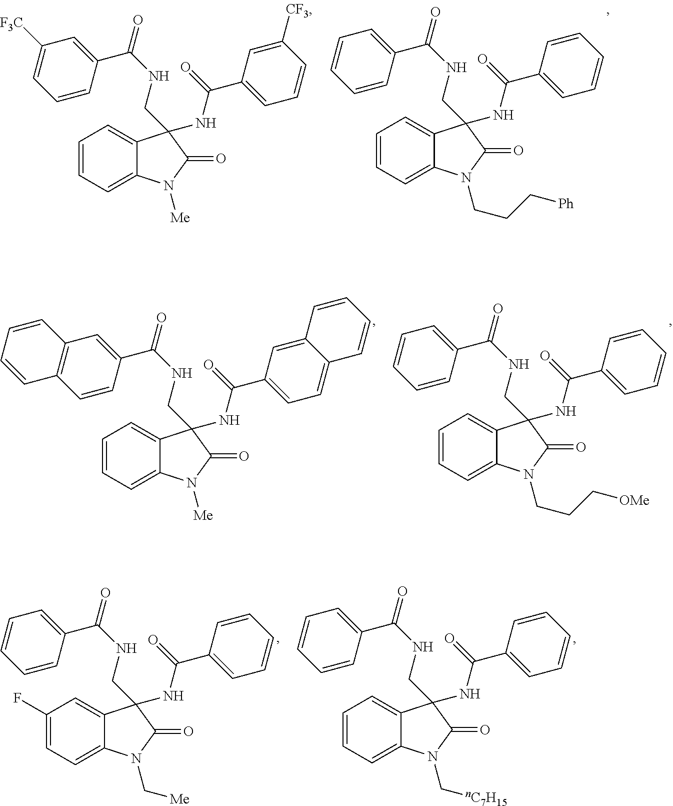

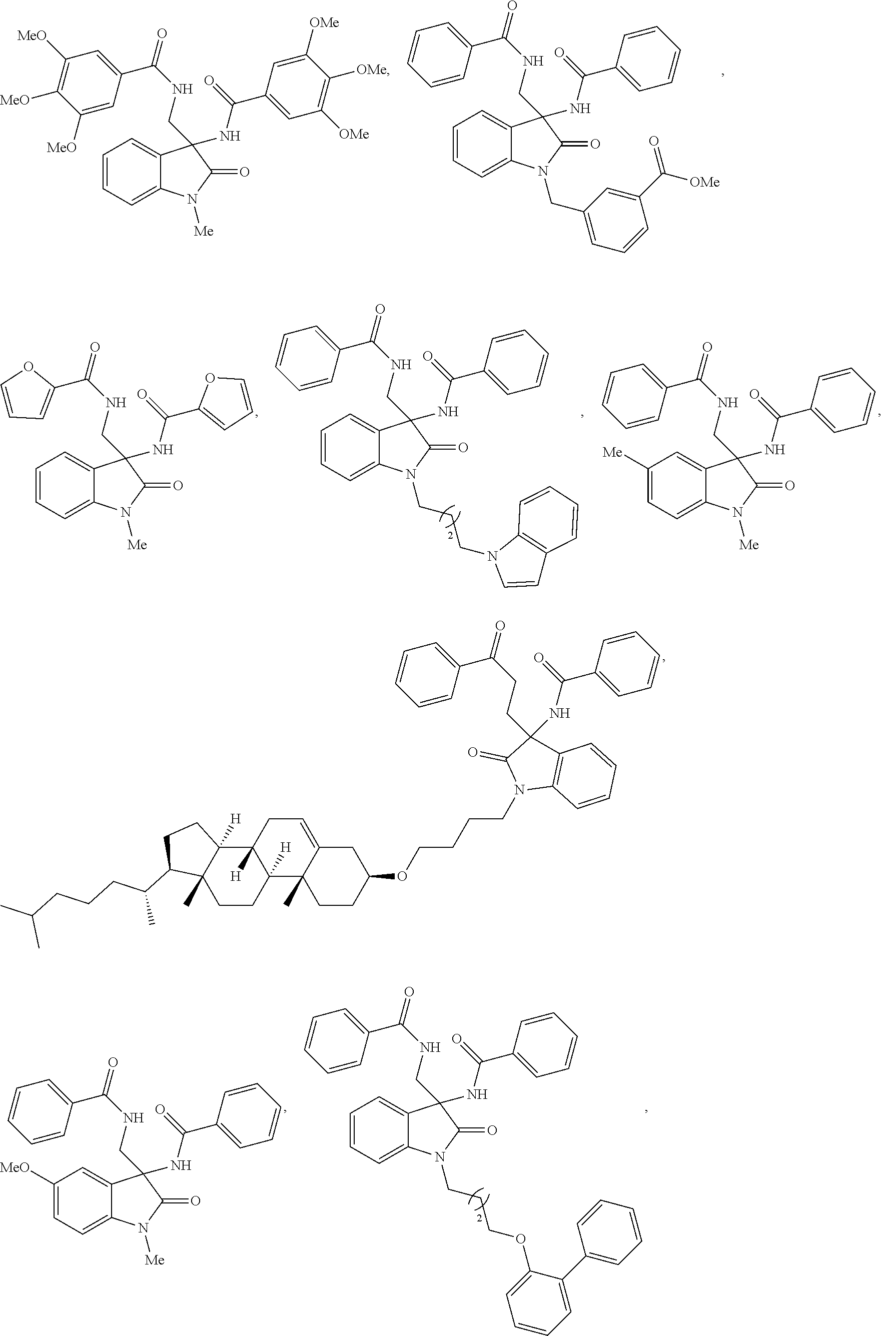

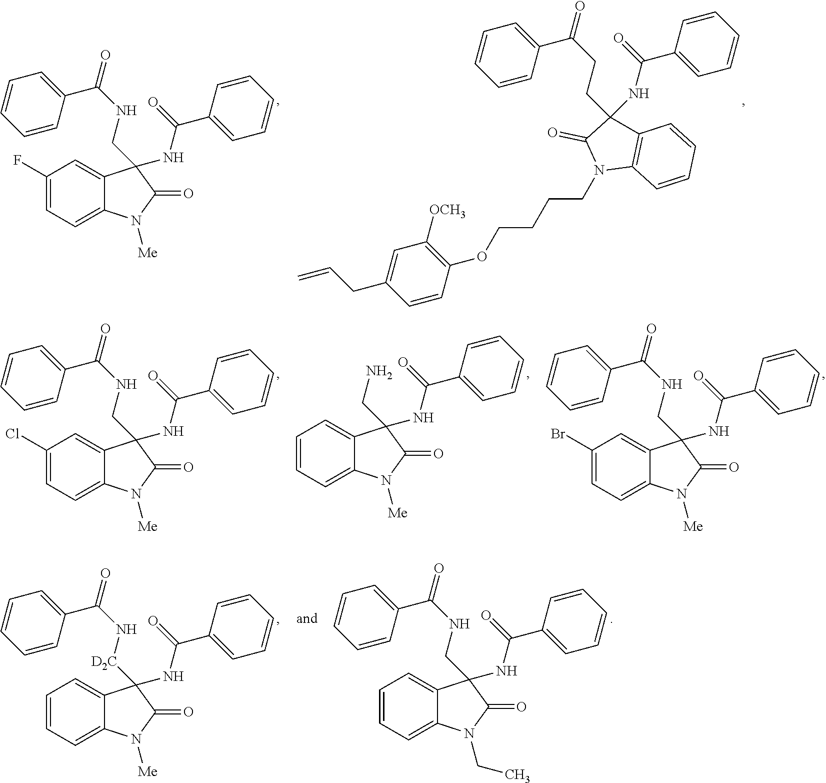

3. A process of for preparing the compound of formula (I), or the isomer, solvate, or tautomer form thereof comprising the steps of:
i. reacting a compound of formula (II) with a compound of formula (III), 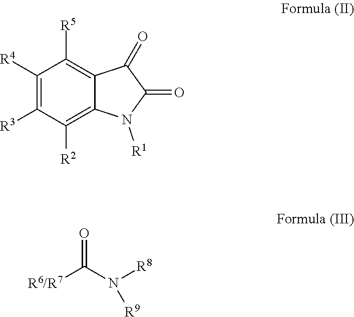
wherein
R¹, R², R³, R⁴, and R⁵, R⁶ and R⁷ are claimed in claim 1;
R⁸ is hydrogen, alkoxy, deuterium or deuterated alkoxy, and
R⁹ is hydrogen, alkoxy, or deuterated alkoxy
in a solvent in presence of a base and Fe catalyst to obtain the compound of formula (I).

4. The process as claimed in claim 3, wherein the base is selected from the group consisting of lithium tert-butoxide (LiO^{t}Bu), potassium tert-butoxide (KO^{t}Bu), sodium tert-butoxide (NaO^{t}Bu), sodium carbonate (Na₂CO₃), potassium carbonate (K₂CO₃), tripotassium phosphate (K₃PO₄), lithium acetate (LiOAc) and sodium acetate (NaOAc).

5. The process as claimed in claim 3, wherein the solvent is selected from the group consisting of toluene, para-xylene, tert-butyl benzene and 1,4-dioxane.

6. The process as claimed in claim 3, wherein the Fe catalyst is selected from the group consisting of Iron(III) chloride (FeCl₃), Iron(II) chloride (FeCl₂), Iron(III) bromide (FeBr₃), Iron(II) bromide (FeBr₂), Iron(II) acetate [Fe(OAc)₂], Iron(III) acetate [Fe(OAc)₃] and Tris(acetylacetonato)iron(III) [Fe(acac)₃].

7. The process as claimed in claim 3, wherein the process step (i) is carried out at a temperature in a range of 100° C. to 120° C. for a time period in the range of 12 hrs to 24 hrs.

8. The process as claimed in claim 3, wherein yield of the compound is in range of 85-95%.

9. (canceled)
